(19) United States
(12) Patent Application Publication
(10) Pub. No.: US 2024/0408071 A1
(43) Pub. Date: Dec. 12, 2024

(54) NOVEL COMBINATION OF SEROTONIN RECEPTOR (5-HTR2B) ANTAGONIST AND AN IMMUNOMODULATOR AND CHEMOTHERAPEUTIC DRUGS FOR INHIBITION OF CANCER

(71) Applicant: NATIONAL CENTRE FOR CELL SCIENCE, Pune, Maharashtra (IN)

(72) Inventors: Girdhari LAL, Pune, Maharashtra (IN); Surojit KARMAKAR, Pune, Maharashtra (IN)

(73) Assignee: NATIONAL CENTRE FOR CELL SCIENCE, Pune, Maharashtra (IN)

(21) Appl. No.: 18/696,916

(22) PCT Filed: Sep. 30, 2022
(86) PCT No.: PCT/IN2022/050873

§ 371 Date: Mar. 28, 2024

(30) Foreign Application Priority Data
Sep. 30, 2021 (IN) 202121044467

(51) Int. Cl. A61K 31/4439; A61K 31/4745; A61K 31/513; A61K 31/555; A61K 39/00; A61P 35/00; C07K 16/28

(52) U.S. Cl. CPC A61K 31/4439; A61K 31/4745; A61K 31/513; A61K 31/555; A61P 35/00; C07K 16/2818; C07K 16/2827; A61K 2039/505

(57) ABSTRACT

The present invention discloses the use of the novel combination comprising HTR_{2B} antagonist and check-point inhibitors/blockers or various classes of chemotherapeutic agents. The present invention also discloses a composition comprising the combination of the present invention. The present combination and composition are found to possess enhanced activity against various cancer, especially tumors, preferably tumors of epithelial origin, selected from the group comprising colon cancer, breast cancer, and melanoma.

### FIELD OF THE INVENTION

The present invention relates to the field of pharmaceuticals. In particular, the present invention is drawn to a combination of an antagonist of serotonin receptor 5-HTR_{2B} with an immunomodulator and a method for using the combination and the use of the said combination to inhibit cancer.

### BACKGROUND OF THE INVENTION

5-HT receptors, 5-hydroxytryptamine receptors, or serotonin receptors, are G protein-coupled receptors and ligand-gated ion channels found in the central and peripheral nervous systems. Among various proteins included in a relatively wide GPCR family, serotonin 5-HT receptors are highly attractive as important biological targets with enormous clinical importance. They mediate both excitatory and inhibitory neurotransmission. The neurotransmitter serotonin is a natural ligand for serotonin receptors (5-HTRs). Serotonin acts through several receptor types and their subtypes. The increasing number of 5-HTRs and their expression patterns in various tissues has made it challenging to unravel the role of multiple 5-HTRs.

At least 15 distinct subtypes of serotonin receptors (5-HTR₁₋₇) are identified. Except for 5-HTR₃. most of the 5-HTRs belong to the G protein-coupled receptor superfamily (GPCRs). The 5-HTR₃ family of receptors (5-HTR_{3A} & 5-HTR_{3B}) forms a ligand-gated non-specific cation channel. and upon activation, it allows entry of cations like like Na⁺, K⁺, Ca²⁺, and Mg²⁺ upon serotonin binding [(Karmakar and Lal (2021) Theranostics 11(11):5296-5312)]. 5-HTR1 receptor subtypes (5-HTR_{1A}, 5-HTR_{1B}, 5-HTR_{1D}, 5-HTR_{1E}, and 5-HTR_{1F}) and 5-HTR5 (5-HTR_{5A} and 5-HTR_{5B}) are coupled with intracellular G protein (Gai/o) that inhibits adenylyl cyclase and subsequently protein kinase A (PKA)/cyclic adenosine monophosphate (cAMP) activity. 5-HTR2 subtype is coupled with intracellular stimulatory G protein (Gq/11) to stimulate intracellular calcium signalling through activation of phospholipase C and also employs the mitogen-activated protein kinase pathway (MAPK). 5-HTR4, 5-HTR6, and 5-HTR7 trigger the PKA/cAMP axis via stimulatory G protein (Gs).

Among different serotonin receptor subtypes, expression of the 5-HTR_{2B} receptor subtype is widespread in the body, and its stimulation triggers a wide range of signalling pathways that include MAPK, phospholipase C/Ca²⁺/calmadoulin, protein kinase C (PKC), and protein kinase A (PKA) and PI3 kinase, mTOR and Notch pathways. However, the length and breadth of the activity of these signals are yet to be determined, and the functions of 5-HTR_{2B} are still being defined.

Human biopsies revealed the serotonin receptor HTR_{2B} expression, correlating with downstream signals, e.g., phosphorylated p70S6K and proliferation. 5-HTRs are present at various tumor stages, and antagonists to these receptors can inhibit the proliferative activity of androgen-independent PC cell lines. (European Urology Volume 47, Issue 6, June 2005, Pages 895-900). In some arts, it is envisaged that targeting the 5-HTR_{2B} receptor may be an effective antiproliferative and antifibrotic strategy for small intestinal neuroendocrine tumors (SI-NETs) because it inhibits tumor microenvironment fibroblasts as well as NET cells. Fibrosis and proliferation appear to be biologically interfaced with neuroendocrine neoplasia domains (J Mol Cell Cardiol. 2018 February; 115: 94-103). However, the approach as discussed in the prior art is different, and the data is inconclusive.

The prior art discussed the role of antagonists of 5-HTR_{2B} for cancer therapy. However, there is no conclusive evidence present in the prior art on the effect of the 5-HTR_{2B} receptor or its antagonists and its effect on cancer. There are also no studies in the art on the effect of immunomodulators in conjunction with 5-HTR_{2B} antagonists. Hence there is a need for providing a treatment using 5 HTR_{2B} antagonists. However, since cancer treatment is very critical, it is necessary to administer a synergistic combination so as to effectively treat cancer when a subject is affected. There is no such synergistic combination involving HTR_{2B} antagonists. Hence there is a need for such synergistic combination.

### OBJECT OF THE INVENTION

An object of the present invention is to provide a novel combination comprising an antagonist of serotonin receptor 5-HTR_{2B} along with an immunomodulator, a composition comprising the combination, a method of treatment using this combination or composition and its use. The present invention also provides a novel combination comprising the antagonist of serotonin receptor 5-HTR_{2B} along with an immunomodulatory and other pre-approved chemotherapeutic drug and a composition comprising the combination, a method of treatment using this combination or a composition and its use.

### SUMMARY OF THE INVENTION

The present invention discloses a novel combination comprising the HTR_{2B} antagonist with an immune modulator. The combination of the present invention acts against cancer, especially tumors including melanoma, colon cancer, and adenocarcinoma. The present invention also discloses a composition comprising the combination of the present invention and a method of treating the cancer. The present invention discloses the use of the novel combination comprising HTR_{2B} antagonist and check-point inhibitors/blockers or various classes of chemotherapeutic agents. The combination and the composition containing the present combination is found to possess enhanced activity against various tumors especially tumors of epithelial origin.

### BRIEF DESCRIPTION OF THE FIGURES

FIG. 1 depicts the correlation between the expression of 5-HTR_{2B} and the survival of various cancer subjects: TCGA database of subjects with tumors of various tissue origins was analysed using the online platform oncolnc.org. Kaplan-Meier plot of indicated tumor subjects between the highest 5-HTR_{2B}-expressing (top 25%) subject survival compared to subjects with the lowest expression of 5-HTR_{2B} (bottom 25%). The P-value shown was calculated using the Log-rank test between high and low 5-HTR_{2B} expressing groups. n=number of subject samples used in the analysis.

FIG. 2 depicts that the expression of 5-HTR_{2A} and 5-HTR_{2B} negatively correlates with the survival of COAD subjects: (A) COAD tumor data from the TCGA database was analysed using the online platform oncolnc.org. Kaplan-Meier plot of COAD subjects from the TCGA database showing the subjects with the highest expression of 5-HTR_{2B} (top 25%; n=110; indicated by red line) show the lowest survival rate as compared to subjects with the lowest expression of 5-HTR_{2B} (bottom 25%; n=110; indicated by blue line). (B) Immuno-Histochemistry data of human colon tumor microarray tissue showing expression of 5-HTR_{2B} (green) and Ki67 (red) in the colon (lamina propria) and lamina propria of colon adenocarcinoma (COAD) tissue (right) and tumor segment of COAD tissue (left). Images are taken in 200× magnification and inset images are acquired in 600× magnification and DAPI (blue) is used as the counterstain. (C) Immuno-Histochemistry data of human colon tumor microarray tissue showing expression of TPH1 (brown) in the colon (lamina propria) and lamina propria of colon adenocarcinoma (COAD) tissue (right) and tumor portion of COAD tissue (left). Images are taken in 200× magnification and inset images are acquired in 600× magnification and Haematoxylin (blue) is used as a nuclear stain. (D) Immuno-Histochemistry data of human colon tumor microarray tissue showing expression of serotonin (blue) and neurofilament-H (red) in the control colon (lamina propria) and lamina propria of colon adenocarcinoma (COAD) tissue (right) and tumor portion of COAD tissue (left). Images are taken in 200× magnification and inset images are acquired in 600× magnification and DAPI (cyan) is used as counterstain.

FIG. 3 depicts a mouse colon tumor showing an expression of 5-HTR_{2B} and TPH1: (A) MC38 cells were injected into C57BL/6 mice, and tumors were grown. Tumors were also harvested at seven days intervals and IHC was performed on the cryo-sectioned tumor tissues for various components of the serotonergic system. (B) Immunohistochemistry of murine MC38 tumor tissue from days 7, Day 14, and day 21 after tumor inoculation showed variable expression of TPH1, serotonin, and 5-HTR_{2B}. The nucleus is stained with DAPI. All the images in the panel taken in 600× magnification and a 4× zoomed image of the original magnification image are shown in the inset. (C) The serum was collected at every seven-day interval either from naïve mice (n=8 mice) or mice after tumor inoculation (n=8 mice), and a competitive ELISA for serotonin was performed. The concentration of serum serotonin is plotted with time. Error bar represents ±standard error of the mean (SEM).

FIG. 4 depicts Colon cancer (MC38) and shows an expression of serotonergic receptors and TPH1: (A-B) Total cDNA was prepared from MC38 cells, and semi-quantitative PCR was performed for different subtypes of serotonin receptors. The amplification of various 5-HTRs was analysed using an agarose gel electrophoresis and their relative expression compared to internal control (GAPDH) is plotted. (C) MC38 cells were also stained with intracellular TPH1 and imaged in a confocal microscope at 600× magnification. The single cell image in the inset was taken at 1000× magnification. DAPI is used as a counterstain. (D-E) Relative mRNA expression of different serotonin receptor subtypes and TPH1, DDC, and MAO-A in MC38 cells were measured by qRT-PCR and their relative expression values were plotted.

FIG. 5: Different immune cell subtypes show variable expression of components of the serotonergic system: (A-D) Normalized relative mRNA expression (ΔΔCq) of 5-HTR_{2B}, 5-HTR_{3A}, 5-HTR₇, and TPH1 in sorted different immune cells was measured by qRT-PCR and plotted. (E) Relative expression of TPH1, DDC, and MAO-A in vitro differentiated CD4 T cells. Here the values are shown as fold change as compared with naïve CD4 T cells. (F) Relative expression of 5-HTR_{2B} in vitro differentiated CD4 T cells. Here the values are shown as fold change as compared with naïve CD4 T cells. (G-H) Expression of 5-HTR2B on different subtypes of CD4 T cells in vivo was measured by flow cytometry and their MFI values are overlayed and their statistical comparison was plotted. (I-K) Relative expression of 5-HTR_{2B}, TPH1, and MAO-A in mice naïve or in vitro activated CD8 T cells in the presence of anti-CD3 and anti-CD28 antibodies and either in the presence or absence of IL-2. Here the values are shown as a fold change as compared to naïve CD8 T cells. (L-M) Expression of 5-HTR_{2B} on different subtypes of CD8 T cells in vivo was measured by flow cytometry and their MFI values are overlayed and their statistical comparison was plotted. Error bar represents t standard error of the mean (SEM).

FIG. 6 depicts that 5-HTR2B signalling in colon cancer affects cell proliferation and viability: Colon cancer MC38 cells were treated with serotonin (100 μM), 5-HTR_{2B} agonist (BW-723C86; 50 μM), or 5-HTR_{2B} antagonist (SB-215505; 50 μM) for 48 hours, and cell cycle analysis was performed by propidium iodide staining using flow cytometry. MC38 cells were also treated with a graded dose of 5-HTR_{2B} agonist (BW-723C86) or serotonin. MTT assay was performed to measure cell viability. (A) After treatment with 5-HTR_{2B} agonist BW-723C86, serotonin, or 5-HTR_{2B} antagonist SB-215505, the relative cell cycle analysis is shown. (B) Relative cell viability data of MC38 cells following treatment of graded doses of 5-HTR_{2B} agonist BW-723C86 or serotonin are shown. The data shown are representative of one of the three independent experiments. (C) MC38 tumor sections from the control tumor and 5-HTR_{2B} antagonist treated tumor were stained for CD31 and the number of CD31⁺ micro-blood vessels/mm² of tissue sections was counted and plotted to observe angiogenesis. Error bar represents ±standard error of the mean (SEM).

FIG. 7 depicts that antagonizing the 5-HTR_{2B} in mice inhibits breast and colon tumor growth: (A) MC38 colon cancer cells were injected subcutaneously into the right flank of C57BL/6 mice. After seven days when tumors became palpable, mice were treated with 5-HTR_{2B} antagonist SB-215505 (n=5 mice; 4 mg/kg/day), 5-HTR_{2B} antagonist RS-127445 (n=5 mice; 4 mg/kg/day) 5-HTR_{2A} antagonist ritanserin (n=5 mice; 4 mg/kg/day), pan-5-HTR₂ antagonist ketanserin (n=5 mice; 4 mg/kg/day) or left untreated and used as control (n=5 mice). Tumor volumes were measured and plotted. (B) MC38 colon cancer cells were injected subcutaneously into the right flank of C57BL/6 mice. After seven days when tumors became palpable, one group was treated with 5-HTR₇ antagonist SB-269970 (n=6 mice; 3 mg/kg/day) or left untreated (n=6 mice). Tumor volume was measured and plotted. (C) After seven days of the setting of tumor growth, one group was treated with a serotonin transporter blocker (SSRI) sertraline hydrochloride (n=6 mice; 15 mg/kg/week) or left untreated (n=6 mice). Tumor volume was measured and plotted. (D) C57BL/6 mice were given subcutaneously injected MC38 cells. After seven days of tumor growth, one group of mice was treated with different doses of 5-HTR_{2B} antagonist SB-215505 (n=6). Tumor volume was measured and plotted. (E) C57BL/6 mice were given subcutaneously injected MC38 cells. After seven days of the tumor growth, one group of mice was treated with different doses of 5-HTR2B antagonist SB-215505 (n=6), one group treated with 5-HTR_{2B} agonist BW-723C86 (n=6; 4 mg/kg/day). Tumor volume was measured and plotted. (F) MC38 cells were subcutaneously injected into C57BL/6 mice. After seven days when the tumor became palpable in mice, one group of mice was treated with FDA-approved 5-HTR_{2B} antagonist cariprazine (Carispec; Sun pharma) via oral gavage (n=5 mice; dose 5 mg/kg body weight/day) or left untreated (n=5 mice). Tumor growth was measured and plotted. (G) Breast cancer cell lines (4T1-Luc cells) were injected into the mammary fatpad of BALB/c mice. After seven days of the tumor growth setting in mice, one group of mice was treated with 5-HTR_{2B} antagonist SB-215505(n=5 mice) or left untreated (n=5 mice). Tumour volumes were measured and plotted. (H) Mice melanoma (B16F10 cells) were injected subcutaneously in the left flank of C57BL/6J mice. After seven days of the tumor growth setting in mice, one group of mice was treated with 5-HTR_{2B} antagonist SB-215505(n=5 mice) or left untreated (n=5 mice). Tumour volumes were measured and plotted. (I) Statistical analysis of the flow cytometric data showing the differential expression of F4/80⁺CD11B⁺ macrophages within MC38 tumor following treatment with 5-HTR_{2B} antagonist. (J) Statistical analysis of the flow cytometric data showing the differential expression of F4/80⁺CD11B⁺ iNOS⁺ M1 macrophages within MC38 tumor following treatment 5-HTR_{2B} antagonist. (K) Statistical analysis of the flow cytometric data showing the differential expression of F4/80⁺CD1B⁺ Arg1⁺ M2 macrophages within MC38 tumor following treatment with 5-HTR_{2B} antagonist. (L) Immuno-Histochemistry data of MC38 tumor tissue from control and 5-HTR_{2B} antagonist treated mice showing expression of F4/80 (cyan), iNOS (blue), arginase 1 (green), and CD11c (red). Images are taken in 20× magnification and inset images are acquired in 600× magnification and DAPI (blue) is used as counterstain. All the images in the panel were taken at 200× magnification. The image in the inset was taken in 600× magnification. All the statistical analysis was performed using Student's ‘t’ test or two-way ANOVA using Sidak's multiple comparison test. *P<0.05; **P<0.01; ***P<0.00; ****P<0.0001 Error bar represents mean±standard error of means (SEM).

FIG. 8 depicts that 5-HTR_{2B} antagonism promotes antigen-specific immune response to the tumor: (A) Experimental protocol for SB-215505 treatment in MC38 tumor-bearing NRG mice. In brief, (B) MC38 cells were injected subcutaneously into NRG mice. After seven days of the tumor growth setting in mice, one group of mice was treated with 5-HTR_{2B} antagonist SB-215505 (n=5 mice) or left untreated (n=5 mice). Tumour volumes were measured and plotted. (C) Experimental protocol for adoptive transfer of SB-215505 pre-treated naïve or MC38 tumor primed immune cells in MC38 tumor-bearing NRG mice. In brief: (D) MC38 cells were injected subcutaneously into NRG mice. Those mice were adoptively transferred with naïve (n=4 mice) or MC38 tumor primed immune cells (n=4 mice) or SB-215505 pre-treated naïve (n=4 mice) or SB-215505 pre-treated MC38 tumor primed immune cells (n=4 mice) from the day of tumor inoculation. After the tumor begins to appear, tumor volumes were measured and plotted. Error bar represents tstandard error of means (SEM)

FIG. 9 depicts the effect of5-HTR antagonists on the CTL response MC38 tumor model in mice: (A) Clinical data correlation study. A TIDE computational method was used to study the association between the tumor-infiltrating CD8 T cells (also known as cytotoxic T lymphocytes, CTL) level and overall subject survival in relation to the intratumoral 5-HTR_{2B} gene expression level. For each patient cohort, tumor samples were divided into 5-HTR_{2B}-high (samples with 5-HTR_{2B} expression one standard deviation above the average; shown in left survival plot) and 5-HTR_{2B}-low (remaining samples; shown in the right survival plot) groups, followed by analyzing the association between CTL levels and survival outcomes in each group. The CTL levels were estimated as the average expression levels of CD8A, CD8B, GZMA, GZMB, and PRF. Each survival plot presented tumors in two subgroups: the CTL-High group (red) had above-average CTL values among all samples, whereas the CTL-low group (blue) had below-average CTL values. (B-C) Flow cytometric data and statistical analysis of the percentage of intra-tumoral CD69⁺ activated CD8 population from the control and RS-127445-treated MC38 tumor-bearing mice. (D-E) Flow cytometric data and statistical analysis of the percentage of intra-tumoral IFN-γ⁺ CD8 T cell population from the control and RS-127445 treated MC38 tumor-bearing mice. (F-G) Histogram overlay and statistical data comparison showing relative MFI of IFN-γ on CD8a⁺ T cells from MC38 tumor of control and RS-127445 treated mice (H-I) Flow cytometric data and statistical analysis of the percentage of intra-tumoral IFNγ⁺ Granzyme B⁺ CD8 T cell population from the control and RS-127445 treated MC38 tumor-bearing mice. (J-K) Histogram overlay and statistical data comparison showing relative MFI of granzyme B on IFN-γ⁺CD8a⁺ T cells from MC38 tumor of control and RS-127445 treated mice. (L-N) Flow cytometric data and statistical analysis of the percentage of intra-tumoral CD44⁺CCR7⁺ central memory CD8 T cell population and CD44⁺CCR7⁻ effector CD8 T cell populations from the control and RS-127445 treated MC38 tumor-bearing mice

FIG. 10 depicts CD8 T cell-mediated immunity is responsible for the 5-HTR_{2B} antagonist-mediated anti-tumor response: (A) CD8 T cell was depleted in C57BL/6J mice using anti-CD8 monoclonal antibody and inoculated with MC38 cells. Then those mice were treated with either RS-127445 (5 mg/kg/day) from the seventh day of tumor inoculation or left untreated. Tumor volume was measured and plotted. (B) Flow cytometric data showing the CD8 depletion in the inguinal lymph node. (D-E) Flow cytometric data and statistical analysis of the percentage of intra-tumoral IFN-γ⁺CD4 T cell population from the control and RS-127445 treated MC38 tumor-bearing mice. (F-G) Flow cytometric data and statistical analysis of the percentage of intra-tumoral IL-10⁺ CD4 T cell population from the control and RS-127445 treated MC38 tumor-bearing mice. (H) Serum IL-10 ELISA of the serum samples from the control and RS-127445 treated MC38 tumor-bearing mice. (I) Serum TNF-alpha ELISA of the serum samples from the control and RS-127445 treated MC38 tumor-bearing mice. All the statistical analysis was performed using either Student's ‘t’ test with Tukey's multiple comparison test or two-way ANOVA using Sidak's multiple comparison test. *P<0.05; **P<0.01; ***P<0.00; ****P<0.0001 Error bar represents mean±standard error of means (SEM).

FIG. 11 depicts the effect of 5-HTR antagonists on the antigen-specific CTL response MC38 tumor model in mice: (A) Representative experimental plan of the adoptive transfer experiment where NRG mice. In brief, NRG mice were inoculated with MC38 on one flank and B16F10 cells on the other. These mice received total lymph node cells from naïve C57BL/6J mice or MC38 tumor-bearing control or RS-127445 treated mice. (B) Tumor volume data from the above-described experiment was measured and plotted (C) Immunocytochemistry data showing expression of H2-Kb-SIINFEKL⁺ (red) on MC38 cells transfected with pCL-neo-OVA plasmid on the right side and MC38 cells without transfection. DAPI is used as a counterstain. (D-E) C7BL/6J mice were inoculated with an MC38-ova cell line and treated with RS-127445 or left untreated. After 10 days of such treatment, Total lymph node cells were harvested tagged with CFSE, and cultured in the presence of ovalbumin protein (50 μg/ml) for 5 days. After 5 days cells were checked for CFSE expression by flow cytometry to check proliferation. Here is the data (D) showing the CFSE in CD8⁺ cells. The statistical data of the total percentage of CFSE⁻ CD8 T cells corresponding with the percentage of proliferated CD8 T cells were plotted. (F-G) Flow cytometric data and statistical analysis of the percentage of intra-tumoral H-2Kb⁻ SIINFEKL tetramer⁺ CD8 population from the control and RS-127445 treated MC38-ova tumor-bearing mice. (H-I) Flow cytometric data and statistical analysis of the percentage of intra-tumoral H-2Kb⁻ SIINFEKL tetramer⁺CD69⁺ activated CD8 population from the control and RS-127445 treated MC38 tumor-bearing mice. (J-K) Flow cytometric data and statistical analysis of the percentage of intra-tumoral H-2K^{b}-SIINFEKL tetramer⁺IFN-γ⁺Granzyme B⁺CD8 T cell population from the control and RS-127445 treated MC38 tumor-bearing mice. (D-E) Histogram overlay and statistical data comparison showing relative MFI of granzyme B on IFN-γ⁺ OT-I-tetramer⁺ CD8a⁺ T cells from MC38-ova tumor of control and RS-127445 treated mice. The data shown are representative of one of the two independent experiments. All the statistical analysis was performed using either Student's t-test or two-way ANOVA using Sidak's multiple comparison test. *P<0.05; **P<0.01; ***P<0.00; ****P<0.0001 Error bar represents mean±standard error of means (SEM).

FIG. 12 depicts that 5-HTR_{2B} signalling affects the antigen-specific proliferation of CD4 T cells: (A-B) Total CD4 T cells were harvested from the lymph node of OT-II mice and they were tagged with CFSE and cultured in vitro in the presence or absence of OT-II peptide (Ova₃₂₃₋₃₃₉), IL-2, 5-HTR_{2B} agonist BW-723C86 (10 μM), Serotonin (20 μM) or 5-HTR_{2B} antagonist RS-127445 (15 μM) for 5 days. After 5 days the proliferation of OT-II tetramer⁺ CD4 T cells was measured and plotted as histogram overlay (A) or as statistical data (B). (C-D) Flow cytometric data and statistical analysis of the percentage of intra-tumoral OT-II-tetramer⁺ IFNγ⁺ CD4 T cell population from the control and RS-127445 treated MC38-ova tumor-bearing mice. (E-F) Flow cytometric data and statistical analysis of the percentage of intra-tumoral OT-II-tetramer⁺ IFNγ⁺ CD4 T cell population from the control and RS-127445 treated MC38-ova tumor-bearing mice. (G) Histogram overlay and statistical data comparison showing relative MFI of IL-10 on OT-II-tetramer⁺ CD4⁺ T cells from MC38-ova tumor of control and RS-127445 treated mice. All the statistical analysis was performed using either Student's ‘t’ test or two-way ANOVA using Sidak's multiple comparison test. *P<0.05; **P<0.01; ***P<0.00; ****P<0.0001 Error bar represents mean±standard error of means (SEM).

FIG. 13 depicts 5-HTR_{2B} signaling promotes PD-L1 expression on the MC38 cell line in vitro: (A-B) MC38 cells are treated with 5-HTR_{2B} agonist (BW-723C86-50 μM), serotonin (100 μM), and 5-HTR_{2B} antagonist (RS-127445-50 μM) for 72 hours, and the PD-L1 expression was checked by flow cytometry and the percentage of PD-L1⁺ MC38 cells was plotted. (C-D) MFI of PD-L1 from the above experiment were plotted as histogram overlay (C) or as statistical data points (D). (E) Relative PD-L1 mRNA expression in MC38 cells following BW-723C86 and RS-127445 treatment for 48 hours. Here the values are shown as a fold change as compared to control MC38 cells. (F) Serum PD-L1 level was measured by ELISA of naïve C57BL/6J mice, control MC38 tumor-bearing mice, BW-723C86 (5 mg/kg/day) treated MC38 tumor-bearing mice and RS-127445 (5 mg/kg/day) treated MC38 tumor-bearing mice after 14 days of tumor inoculation. All the statistical analysis was performed using either Student's ‘t’ test or two-way ANOVA using Sidak's multiple comparison test. *P<0.05; **P<0.01; ***P<0.00; ****P<0.0001 Error bar represents mean±standard error of means (SEM).

FIG. 14 depicts the effect of other 5-HTR antagonists on PD-L1 expression on the macrophages or DCs in the MC38 tumor: (A) C57BL/6J mice were injected with MC38 cells and treated with two different 5-HTR_{2B} antagonists (RS-127445 or SB-215505) after 5 days of tumor inoculation or left untreated. Mice were sacrificed on day 24, tumors were harvested, and single-cell suspensions were made. Flow cytometric data acquired from tumor tumor-infiltrating in MC38 tumor using different markers such as CD1 lb, F4/80, CD1 Ic, Gr1, iNOS, Arginasel, and PD-L1 following treatment with 5-HTR_{2B} antagonist SB-215505 and RS-127445. Dimensionality reduction by tSNE plot was prepared using FCS express software. The bottom panel shows various cell types that were represented in the plot. (C) The percentage of CD11b⁺Gr1⁺ monocyte-derived suppressor cells (MDSCs) in the tumor were analyzed and plotted. (D) The percentage of PD-L1 expression on F4/80⁺CD11b⁺ macrophages in a tumor. (E) The percentage of PD-L1 on F4/80⁺CD11b⁺iNOS⁺ M1 macrophages in the tumor. (F) The percentage of PD-L1 on F4/80⁺CD11b⁺Arg1⁺ M2 macrophages in the tumors. (G) The percentage of PD-L1 on CD11c⁺ dendritic cells in the tumors. Each symbol represents data from an individual mouse (Right). All the statistical analysis was performed using either Student's ‘t’ test or two-way ANOVA using Sidak's multiple comparison test. *P<0.05; **P<0.01; ***P<0.00; ****P<0.0001 Error bar represents mean±standard error of means (SEM).

FIG. 15 depicts the effect of the 5-HTR_{2B} antagonist in the combination of anti-PD1 antibodies on MC38 tumor growth in mice: (A) The TIDE analysis shows the comparative overall survival rate of melanoma subjects treated with α-PD1 mAb grouped into 5-HTR_{2B} high (red) and 5-HTR_{2B} (low) z=2.59, P=0.00955. A positive Z score indicates that the expression of 5-HTR_{2B} is negatively correlated with the therapeutic outcome and the P value indicates the expression of 5-HTR_{2B}-low and 5-HTR_{2B}-high groups and calculated by a two-sided Wald test in a Cox- PH regression. (B) MC38 colon cancer cells were injected subcutaneously into the flank of C57BL/6 mice. After seven days of the setting of tumor growth, one group received 5-HTR_{2B} antagonist RS-127445 (n=5 mice) at a dose of 2 mg/kg/day; one group received anti-mouse anti-PD1 antibody (Equivalent to humanized anti-PD1 monoclonal antibodies such as Pembrolizumab, Nivolumab, Cemiplimab, JTX-4014, Spartalizumab, Camrelizumab, Sintilimab, Tislelizumab, Toripalimab, Dostarlimab, INCMGA00012, AMP-224 or AMP-514) (n=5 mice) 50 μg/mice every 4 days; one group received the combination of both (a-PD1+RS-127445) or left untreated (n=5 mice). Tumor volumes were measured and plotted. (C) The tumor weight of the MC38 tumors from the above experiment was measured and plotted. (D-E) Flow cytometric data and statistical analysis of the percentage of intra-tumoral IFNγ⁺Granzyme B⁺ CD8 T cell population from the control and RS-127445 treated, anti-PD1 antibody-treated and combination therapy treated MC38 tumor-bearing mice. (F) MC38 colon cancer cells were injected subcutaneously into the o flank of C57BL/6 mice. After seven days of the setting of tumor growth, one group received 5-HTR_{2B} antagonist RS-127445 (n=5 mice) at a dose of 2 mg/kg/day; one group received anti-mouse anti-CTLA4 antibody (Equivalent to humanized anti-CTLA4 monoclonal antibodies such as Ipilimumab or Tremelimumab)(n=5 mice) 50 μg/mice every 4 days; one group received the combination of both (a-CTLA4+RS-127445) or left untreated (n=5 mice). (G) MC38 colon cancer cells were injected subcutaneously into the flank of C57BL/6 mice. After seven days of the setting of tumor growth, one group received 5-HTR_{2B} antagonist RS-127445 (n=5 mice) at a dose of 2 mg/kg/day; one group received anti-mouse anti-PD-L1 antibody (Equivalent to humanized anti-PD-L1 monoclonal antibodies such as KN035, Atezolizumab, Avelumab, cosibelimab, AUNP12, CA-170 or BMS-986189) (n=5 mice) 50 μg/mice every 4 days; one group received the combination of both (a-PD-L1+RS-127445) or left untreated (n=5 mice). Tumor volumes were measured and plotted. Tumor volumes were measured and plotted. All the statistical analysis was performed using two-way ANOVA using Sidak's multiple comparison test. *P<0.05; **P<0.01; ***P<0.00; ****P<0.0001 Error bar represents mean±standard error of means (SEM).

FIG. 16 depicts the effect of the 5-HTR_{2B} antagonist on the FDA-approved chemotherapy drug treatment: (A) BALB/C mice were inoculated with 4T1-Luc2 cells in the mammary fat pad. After 7 days of tumor inoculation, mice were treated with low doses of paclitaxel (2 mg/kg/alternate day), 5-HTR_{2B} antagonist RS-127445 (2 mg/kg/day), or a combination of both or left untreated. Tumor volume was measured and plotted. (B) MC38 colon cancer cells were injected subcutaneously into the right flank of C57BL/6 mice. After seven days of setting of tumor growth, mice were treated with 5-HTR_{2B} antagonist SB-215505 (n=5 mice; 4 mg/kg/day), 5-fluorouracil (n=5 mice; 10 mg/kg/day), a combination of both (5-fluorouracil and SB-215505) or left untreated (n=5 mice). Tumor growth was monitored and plotted. (C) MC38 colon cancer cells were injected subcutaneously into the right flank of C57BL/6 mice. After seven days of setting of tumor growth, mice were treated with 5-HTR_{2B} antagonist RS-127445 (n=5 mice; 4 mg/kg/day), oxaliplatin (n=5 mice; 4 mg/kg/day), a combination of both (oxaliplatin and RS-127445) or left untreated (n=5 mice). Tumor growth was monitored and plotted. (D) MC38 colon cancer cells were injected subcutaneously into the right flank of C57BL/6 mice. After seven days of setting of tumor growth, mice were treated with 5-HTR_{2B} antagonist RS-127445 (n=5 mice; 4 mg/kg/day), irinotecan (n=5 mice; 15 mg/kg/day), a combination of both (irinotecan and RS-127445) or left untreated (n=5 mice). Tumor growth was monitored and plotted. (E) MC38 colon cancer cells were injected subcutaneously into the right flank of C57BL/6 mice. After seven days of setting of tumor growth, mice were treated with 5-HTR_{2B} antagonist RS-127445 (n=5 mice; 4 mg/kg/day), trifluorothymidine (n=5 mice; 20 mg/kg/day), a combination of both (trifluorothymidine and RS-127445) or left untreated (n=5 mice). Tumor growth was monitored and plotted.

### DETAILED DESCRIPTION OF THE INVENTION

A “subject” herein is a mammal. Preferably the subject herein is a human and includes within its scope a subject.

The present invention discloses a novel combination of 5-HTR_{2B} antagonist with an immunomodulator.

The combination of the present invention may be administered individually or in conjunction with a chemotherapeutic agent. The combination of the present invention possesses enhanced therapeutic efficacy when added as 5-HTR_{2B} antagonist and immunomodulator or along with an anti-cancer agent, especially against the tumors/cancers of epithelial origin, selected from the group comprising melanoma, breast cancer, and colon adenocarcinoma.

In an aspect, the present invention discloses a composition comprising the combination of the present invention. The present invention also discloses the use of the combination of the present invention consisting of a 5-HTR_{2B} receptor antagonist with an immunomodulator as an anti-cancer, or anti-tumor agent. In an aspect, the present invention also discloses a novel combination comprising the antagonist of serotonin receptor 5-HTR_{2B} along with an immunomodulator and other pre-approved chemotherapeutic drugs and the use of the combination for the treatment of cancer.

The present invention is based on the investigation of the inventors during their extensive study pertaining to the expression and modulation of the serotonergic system in the tumor microenvironment. From certain prior art, it can be understood that there exists an expression of various components of the serotonergic system in different cancer like hepatocellular carcinoma, breast cancer, prostate cancer, breast cancer, melanoma, cholangiocarcinoma, etc. ((European journal of medicinal chemistry, 2019, (168), 461-473; European Urology, 2005, (47), 895-900) The Prostate, 2004 (59) 328-336; The journal of urology, 2006 (178) 1648-1653). However, the studies were inconclusive regarding the capacity of cancer cells to synthesize or secrete serotonin. However, such results do not appear to have been exploited for the intervention of cancer or in cancer therapy.

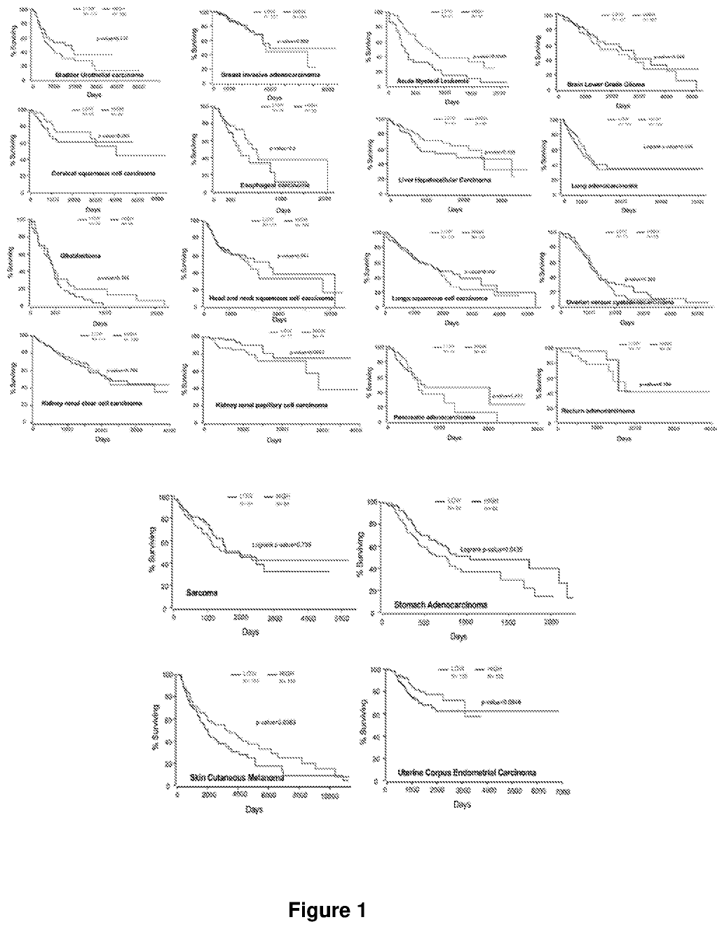

The present invention examines the expression of 5-HTR_{2B} expression in various cancer samples as disclosed by prior art group (Bladder urothelial carcinoma, Cervical squamous cell carcinoma, Glioblastoma, Kidney renal clear cell carcinoma, Breast invasive adenocarcinoma, Esophageal carcinoma, Head and Neck squamous cell carcinoma, Kidney renal papillary cell carcinoma, Acute myeloid leukemia, Liver Hepatocellular Carcinoma, Lung squamous cell carcinoma, Pancreatic adenocarcinoma, Brain lower grade Glioma, Lung adenocarcinoma, Ovarian serous cystadenocarcinoma, Rectal adenocarcinoma, Sarcoma, skin Cutaneous Melanoma, and Uterine Corpus endometrial carcinoma) and its correlation with subjects survival. None of these subjects except for acute myeloid leukemia and stomach adenocarcinoma showed any significant survival with the expression of 5-HTR_{2B} expression (FIG. 1). More importantly the present invention utilises the studies in the intervention of cancer in a synergistic manner. The inventors of the present invention also surprisingly noted that the modulation of the immune system in conjunction with 5-HTR_{2B} antagonists provided synergistic effect in cancer intervention. Also, the present invention discloses that chemotherapy by existing drugs can be potentiated in a synergistic manner by a combination of 5HTR_{2B} antagonists and immunomodulator.

Based on the studies, the present inventors use a novel combination for the intervention of tumors based on a combination of 5HTR_{2B} antagonists with an immunomodulator.

The present invention discloses a combination comprising:
(a) A 5-HTR_{2B} antagonistic compound, and
(b) An immunomodulatory compound

In another aspect, the invention discloses a combination comprising:
(a) A 5-HTR_{2B} antagonistic compound
(b) An immunomodulatory compound, and
(c) Optionally a chemotherapeutic drug

The 5-HTR_{2B} antagonistic compound of the present invention may be selected from the group comprising Agomelatine, Amisulpride, Aripiprazole, Cariprazine, Clozapine, Cyproheptadine, Sarpogrelate, Lisuride, Tegaserod, RS-127,445 (CAS No. 199864-87-4), Metadoxine, SDZ SER-082, Promethazine, EGIS-7625, PRX-08066, SB-200,646, SB-204,741, SB-206,553, SB-215,505 (CAS NO. 162100-15-4), SB-228,357, Terguride, LY-266,097, LY-272,015, preferably the compound is Cariprazine or SB215505 or RS-127445.

The immunomodulatory compound of the present invention may be selected from the group comprising anti-PD1 mAb, anti-PDL1 mAb, and anti-CTLA4 mAb.

The immunomodulatory compound of the present invention may be selected from the group comprising Pembrolizumab, Nivolumab, Cemiplimab, JTX-4014, Sintilimab, Spartalizumab, Camrelizumab, Tislelizumab, Toripalimab, Dostarlimab, INCMGA00012, AMP-224, AMP-514, CD279, Atezolizumab, Avelumab, Durvalumab, CD274, B7-H1, KN035, cosibelimab, AUNP12, CA-170, BMS-986189, Ipilimumab, Tremelimumab, and CD152

The immunomodulatory compound anti-PD1 mAb in mouse and its equivalent that may be selected from the group comprising Pembrolizumab, Nivolumab, Cemiplimab, JTX-4014, Sintilimab, Spartalizumab, Camrelizumab, Tislelizumab, Toripalimab, Dostarlimab, INCMGA00012, AMP-224, AMP-514, and CD279.

The immunomodulatory compound anti-PDL1 mAb in mouse and its equivalent that may be selected from the group comprising Atezolizumab, Avelumab, Durvalumab, CD274, B7-H1, KN035, cosibelimab, AUNP12, CA-170, and BMS-986189.

The immunomodulatory compound anti-CTLA4 mAb in mice and its equivalent that may be selected from the group comprising Ipilimumab, Tremelimumab, and CD152.

The present invention may optionally contain a chemotherapeutic drug. The chemotherapeutic drugs can be selected from the group consisting of drugs that
mimic nucleotides and prevents DNA replication and cell divisions such as 5-fluorouracil (5-FU) and trifluorothymidine;
act as a blocker to cytoskeletal rearrangements which prevent cell adhesion, migration and division such as paclitaxel or taxol;
forms heavy metal adducts with DNA and stall replication and cell division such as oxaliplatin and cisplatin; and
acts as a blocker of topoisomerases that stall the replication fork such as irinotecan.

Any of the drugs involving these pathways can be used as combination therapy with 5-HTR_{2B} antagonist for its anti-cancer formulation.

The chemotherapeutic drug of the present invention may be selected from the group comprising 5-fluorouracil (5-FU), paclitaxel, oxaliplatin, cisplatin trifluorothymidine, and irinotecan.

In the present invention, the 5-HTR_{2B} antagonistic compound is in the range of 30% to 70% by weight, preferably 40% to 50% by weight, and the immunomodulatory compound is in the range of 40% to 80% by weight, preferably 50% to 60% by weight of the total weight of the combination.

In the present invention, the 5-HTR_{2B} antagonistic compound is in the range of 10% to 40% by weight, preferably 15% to 30% by weight, the chemotherapeutic drug, 5-FU is in the range of 60% to 90% by weight, preferably 70% to 85% by weight of the total weight of the combination.

In the present invention, the 5-HTR_{2B} antagonistic compound is in the range of 40% to 60% by weight, preferably 45% to 55% by weight, the chemotherapeutic drug, Paclitaxel is in the range of 40% to 60% by weight, preferably 45% to 55% by weight of the total weight of the combination.

In the present invention, the 5-HTR_{2B} antagonistic compound is in the range of 15% to 50% by weight, preferably 25% to 35% by weight, the chemotherapeutic drug, Oxaliplatin is in the range of 50% to 90% by weight, preferably 65% to 75% by weight of the total weight of the combination.

In the present invention, the 5-HTR_{2B} antagonistic compound is in the range of 5% to 30% by weight, preferably 10% to 20% by weight, the chemotherapeutic drug, Irinotecan is in the range of 70% to 95% by weight, preferably 80% to 90% by weight of the total weight of the combination.

In the present invention, 5-HTR_{2B} antagonistic compound is in the range 1 to 40 mg/kg of body weight, preferably 1 to 20 mg/kg of body weight, more preferably 1 to 5 mg/kg of body weight and most preferably 2 to 4 mg/kg of body weight.

The present invention may contain immunomodulatory compounds in the range 1 to 20 mg/kg of body weight and most preferably 2 to 10 mg/kg of body weight.

The present invention may contain a chemotherapeutic drug, 5-FU in the range 5 to 50 mg/Kg of body weight, preferably 10-25 mg/kg of body weight.

The present invention may contain a chemotherapeutic drug, Paclitaxel in the range 2 to 10 mg/Kg of body weight, preferably 2 to 5 mg/kg of body weight.

The present invention may contain a chemotherapeutic drug, Oxaliplatin in the range 4 tol0 mg/Kg of body weight, preferably 4 to 7 mg/kg of body weight.

The present invention may contain a chemotherapeutic drug, Irinotecan in the range 15 to 50 mg/Kg of body weight, preferably 15 to 30 mg/kg of body weight.

In an embodiment, the present invention discloses a composition comprising combination of 5-HTR_{2B} antagonistic compound with immunomodulatory compound, along with pharmaceutically acceptable excipients.

In yet another embodiment, the present invention discloses a composition comprising combination of 5-HTR_{2B} antagonistic compound with immunomodulatory compound and additionally chemotherapeutic agents, along with pharmaceutically acceptable excipients.

Administration of the compounds of this disclosure, or their pharmaceutically acceptable salts, in pure form or an appropriate pharmaceutical composition, can be carried out via any of the accepted modes of administration or agents for serving similar utilities. Thus, the composition can be administrated as oral, intradermal, transdermal, parenteral, intramuscular, intrathecal, topically, intravaginal, intravesical, intracisternal, or rectally. The composition can be in the form of solid, semi-solid, lyophilized powder, liquid dosage forms or aerosols.

The composition comprising combination of the present invention may be administered orally or via injection at a dose of 0.1 to 500 mg/kg per day. The dose range for adult humans is generally from 5 mg to 2 g/day. Tablets or other forms of presentation provided in discrete units may conveniently contain an amount of one or more compounds that is effective at such dosage or as a multiple of the same, for instance, units containing 5 mg to 500 mg, usually around 10 mg to 200 mg. The number of active ingredients that may be combined with the carrier materials to produce a single dosage form will vary depending upon the host treated and the particular mode of administration.

The present invention also discloses the use of the combination or composition for better reduction of the tumor. The composition comprising combination of the present invention may be administered in a dose of 1 to 100 mg/kg body weight, preferably 1 to 75 mg/kg body weight, more preferably 1 to 50 mg/kg body weight.

The novel combination of the present invention acts synergistically and produces enhanced therapeutic efficacy compared to the individual compounds in reducing the tumor.

The present invention is used for the treatment of cancer cells expressing 5-HTR_{2B} and having higher serotonin synthesizing enzymes (TPH1).

In an embodiment, the present invention discloses a method of treating a subject comprising the administration of
(a) 5-HTR_{2B} antagonistic compound, and
(b) an immunomodulatory compound

either concomitantly or consecutively.

The present invention also discloses a method of treating a subject comprising the administration of
(a) A 5-HTR_{2B} antagonistic compound
(b) An immunomodulatory compound, and
(c) Optionally a chemotherapeutic drug

Without being limited by theory, the present invention showed an increased PD-L1 expression of MC38 tumor cells following 5-HTR_{2B} agonist treatment. Further, in MC38-induced colon tumor-bearing mice, there is an increased free serotonin level in the serum after blocking the reuptake of serotonin using serotonin reuptake inhibitor (SSRI) sertraline which also enhanced the secretory PD-L1 variant level in the serum. This secretory variant of PD-L1 is responsible for most of the resistance to the anti-PD-L1 therapy in the tumor.

In the present invention, it is proposed that the serotonin signalling specifically through 5-HTR_{2B} leads to increased PD-L1 expression by cancer cells. An anti-PD1 antibody, anti-PD-Llor anti-CTLA4 antibody therapy along with a 5-HTR_{2B} antagonist can be used for controlling tumor growth. This may lead to reduced resistance to the anti-PD1 antibody, anti-PD-LIor anti-CTLA4 therapy by cancer and subsequent enhanced response to the immunotherapy. Given the anti-tumor effect of 5-HTR_{2B} alone along with its modulation of an immune response, a low dose of anti-PD1 antibody, anti-PD-L1 or anti-CTLA4 mAb will have low toxicity and much improved anti-tumor activity. 5-fluorouracil (5-FU), Oxaliplatin, Irinotecan, Trifluorothymidine, and paclitaxel have been used as the first line of chemotherapy for many forms of cancers including breast and colorectal cancer. 5-Fluorouracil is one of the first-line medications for colorectal carcinoma. It is an analog of uracil and it gets incorporated in the place of uracil during mRNA synthesis by transcription and the whole transcription machinery halts. Oxaliplatin is a non-targeted anti-neoplastic drug that is used alone or in combination with other drugs in colon cancer. Oxaliplatin causes DNA damage to the cell by forming platinum adducts with the DNA and subsequently halting DNA replication, and transcription and causing reduced cell divisions and apoptosis. Irinotecan is also an FDA-approved chemotherapeutic agent to be used in colorectal cancer that binds to the topoisomerase I-DNA complex and this ternary complex stalls the movement of the replication fork and DNA replication are inhibited. Trifluridine is an approved drug to be used as first-line therapy for CRC. It is a fluorinated pyrimidine nucleoside that inhibits thymidylate synthase irreversibly and also inhibits specific DNA polymerases, necessary for the conversion of dUMP to dTMP in the process of DNA synthesis. Paclitaxel is the first-line therapy for breast cancer where it binds to microtubules and prevents their disassembly, thus preventing the re-assembling of microtubules during cell division, which further inhibits cell division. Based on the present invention, it can be envisaged that a combination of 5-HTR_{2B} antagonists and any of the above-mentioned chemotherapeutic drugs will have potent anti-tumor immunity.

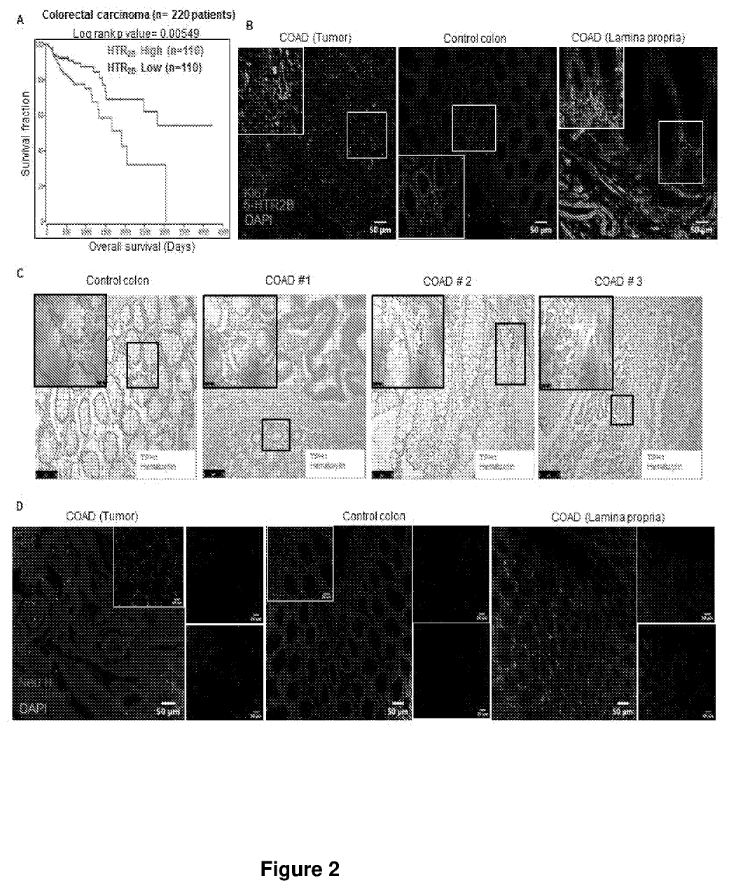

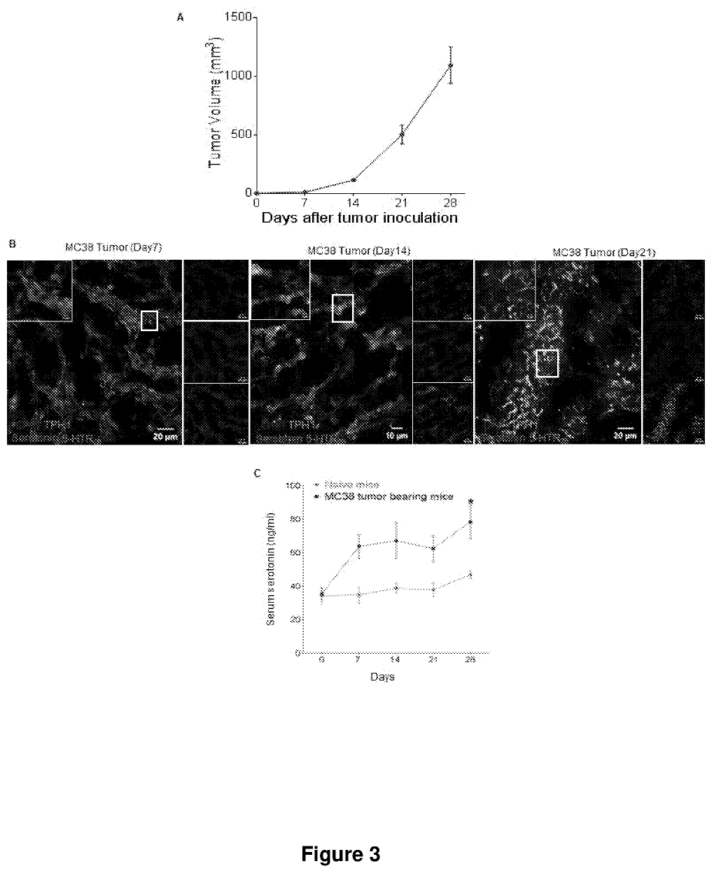

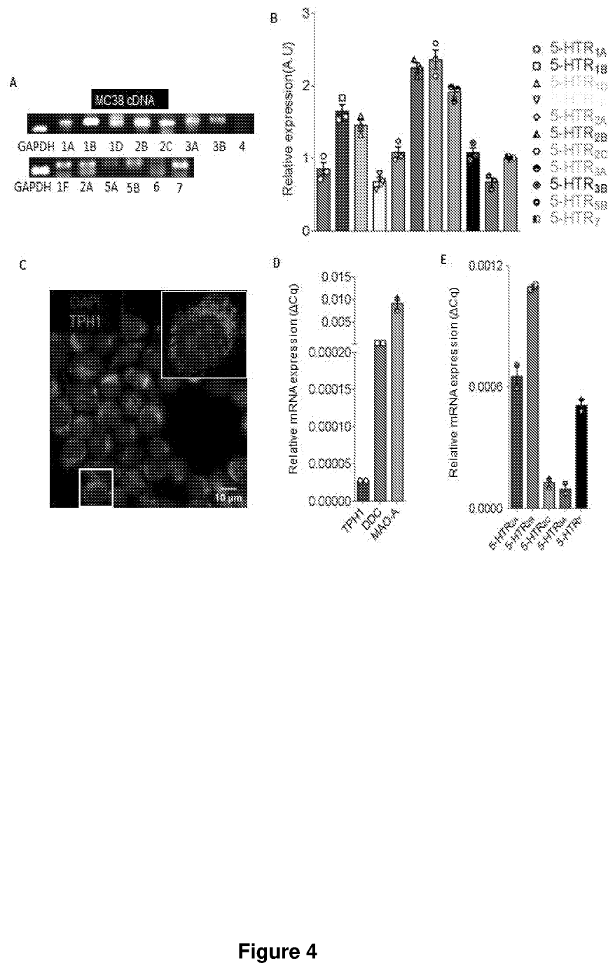

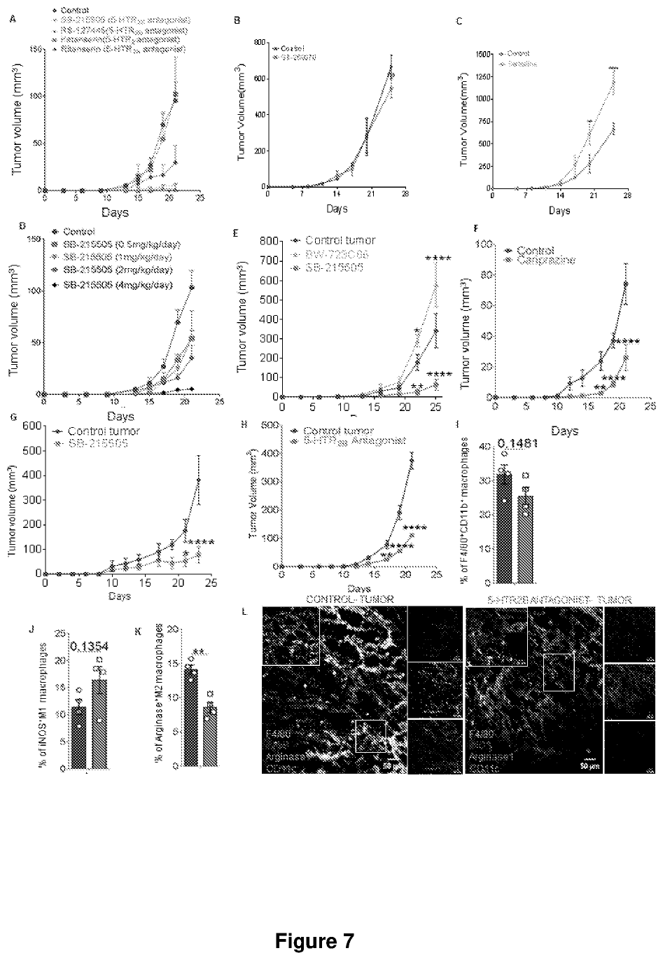

The present inventors demonstrated through extensive research a mouse colon cancer cell line MC38 showed higher expression of serotonin synthesizing enzyme TPH1 (FIG. 4C). This indicates that these cells can synthesize serotonin. Not only in mice colon cancer cell lines but the expression of serotonin synthesizing enzyme (TPH1) is also enhanced in human colon adenocarcinoma tissue sections compared to control colon (FIG. 2C). The synthesis of serotonin is also enhanced in the colon tumor microenvironment compared to the control colon (FIG. 2D). This indicates that in the tumor microenvironment, there is an increased capacity of serotonin synthesis not only by tumor cells but also by tumor-infiltrating immune cells. It was also noted that the expression of serotonin receptor subtype 5-HTR_{2B} was increased in the TME (FIG. 2B). This indicates that not only serotonin synthesis is increased in cancer, but also the responsiveness to the serotonin through a specific receptor is also increased. In the mice, COAD model, the expression of 5-HTR_{2B}, TPH1, and serotonin was increased in larger orthotopic MC38 tumors (FIG. 3B). It was found that the serum serotonin level is higher in the serum of orthotopic MC38 tumor-bearing mice (FIG. 3C). Such increased serum serotonin levels may have resulted from overexpressed TPH1 within MC38 cells, which may contribute to these increased serotonin levels in the serum. But the role of other immune cells in a tumor cannot be ignored as seen in enhanced TPH1 levels in the tumor-infiltrating cells in human colon tumors (FIG. 2C). Modulation of this dysregulated serotonergic system in vivo showed a very promising outcome, as presented in the present application (FIG. 7A-F). It was also observed that chemical antagonists of the 5-HTR_{2B} receptor subtype showed potential in decreasing colon cancer (FIG. 7A, 7D-E) and breast cancer (FIG. 7K) tumor growth. In contrast, the chemical agonist of 5-HTR_{2B} increased tumor growth in the murine colon cancer model (FIG. 7E). This indicates that antagonizing the signaling through 5-HTR_{2B} has an inhibitory effect on tumor growth. 5-HTR_{2B} antagonists reduced the percentage count of tumor-promoting M2 macrophages in the TME, which may contribute to tumor reduction (FIG. 7G-I).

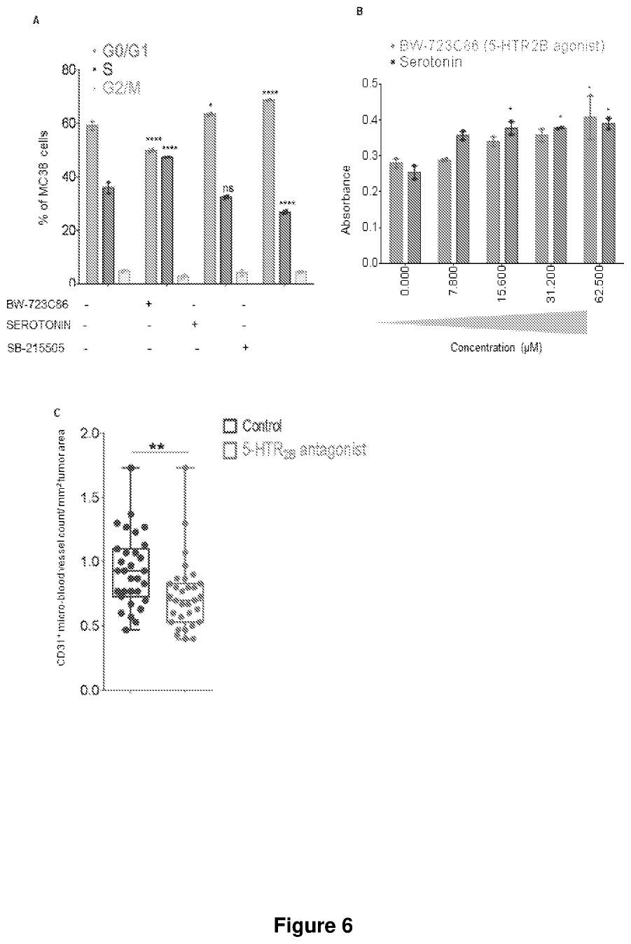

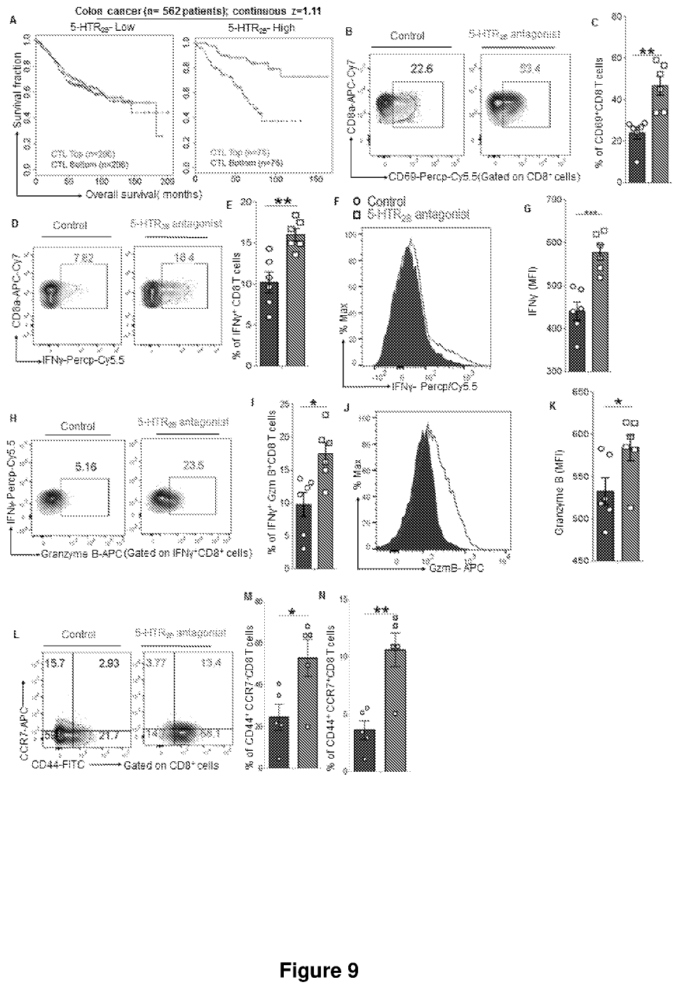

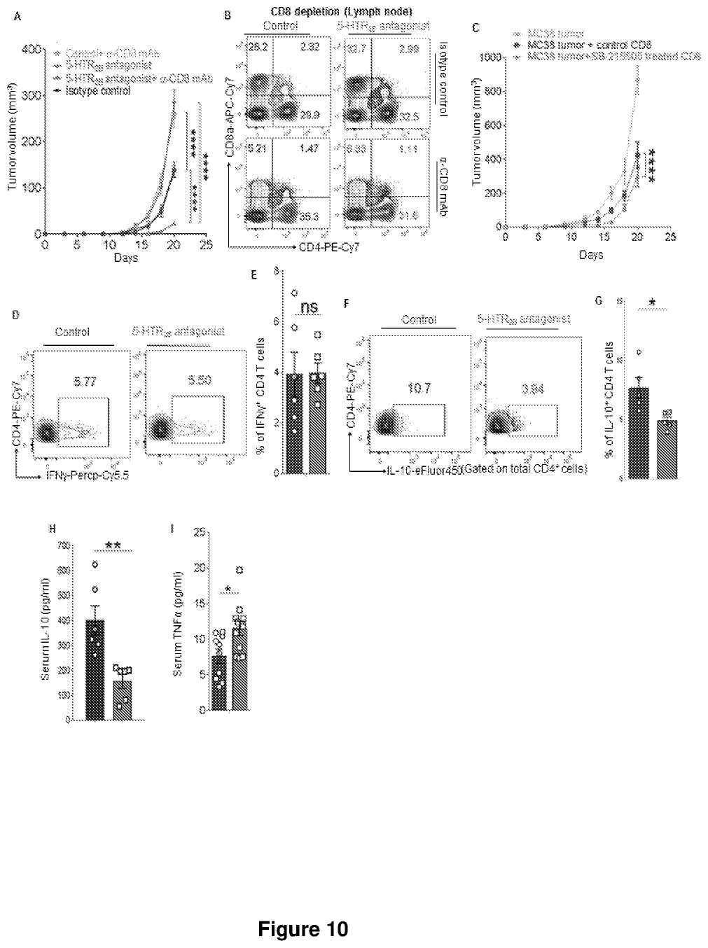

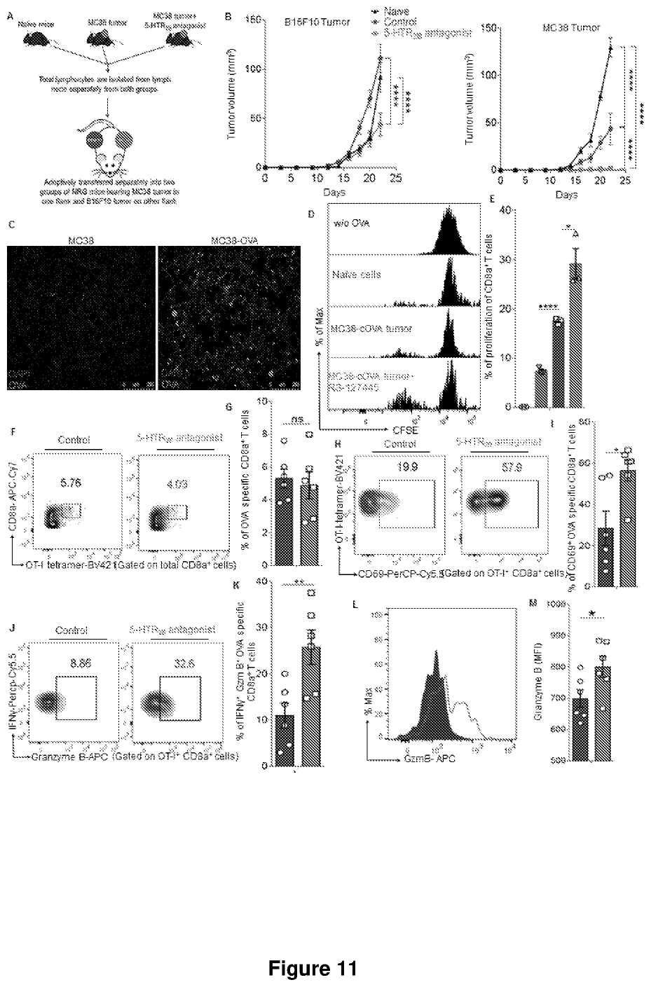

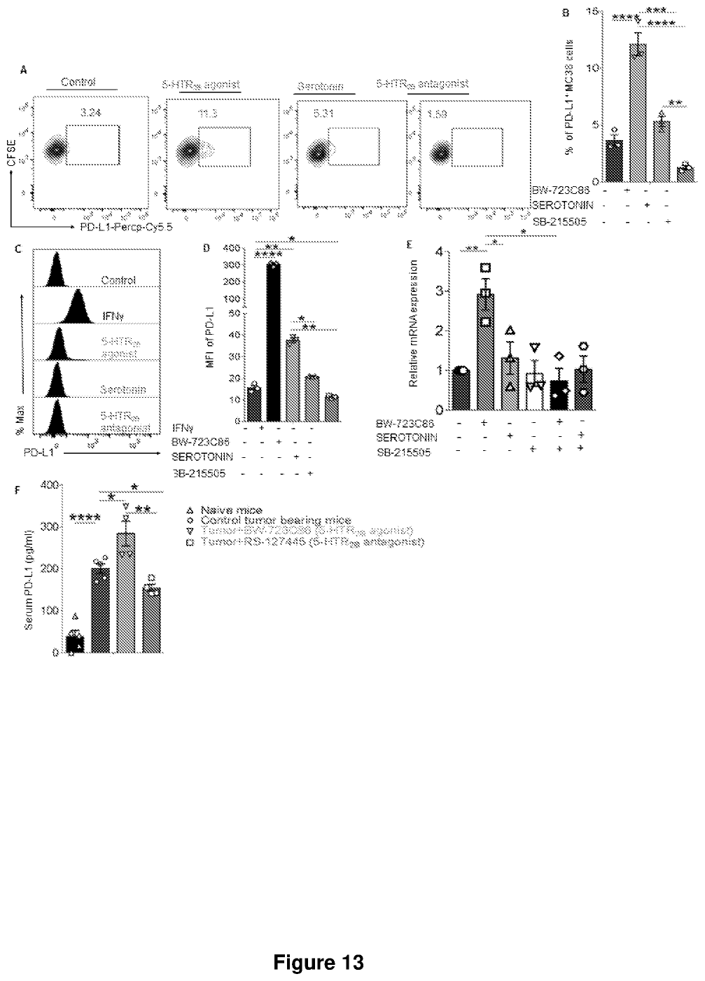

On examining experimentally, and as presented in FIG. 6, in vitro data supports that 5-HTR_{2B} signaling on the murine colon cancer cell line increases cancer cell viability and proliferation capacity. The impact of serotonin signaling on the immunomodulatory capacity of cancer cells has never been studied so far. The present invention evaluates the expression of PD-L1 on MC38 cells following serotonin signaling. It was found that 5-HTR_{2B} signaling promotes the expression of PD-L1 on cancer cells (FIG. 13). This indicates that 5-HTR_{2B} signaling modulates the immuno-modulatory capacity of cancer cells. 5-HTR_{2B} antagonism improves cytotoxic T cell response in the tumor and suppresses regulatory CD4 T cell response in colon cancer (FIG. 9-10). 5-HTR_{2B} antagonism also enhances antigen-specific CTL response in colon tumors (FIG. 11), either due to the direct effect of the 5-HTR_{2B} signaling on CTLs or due to the modulation of the immune-modulatory capacity of cancer or innate cells in the TME.

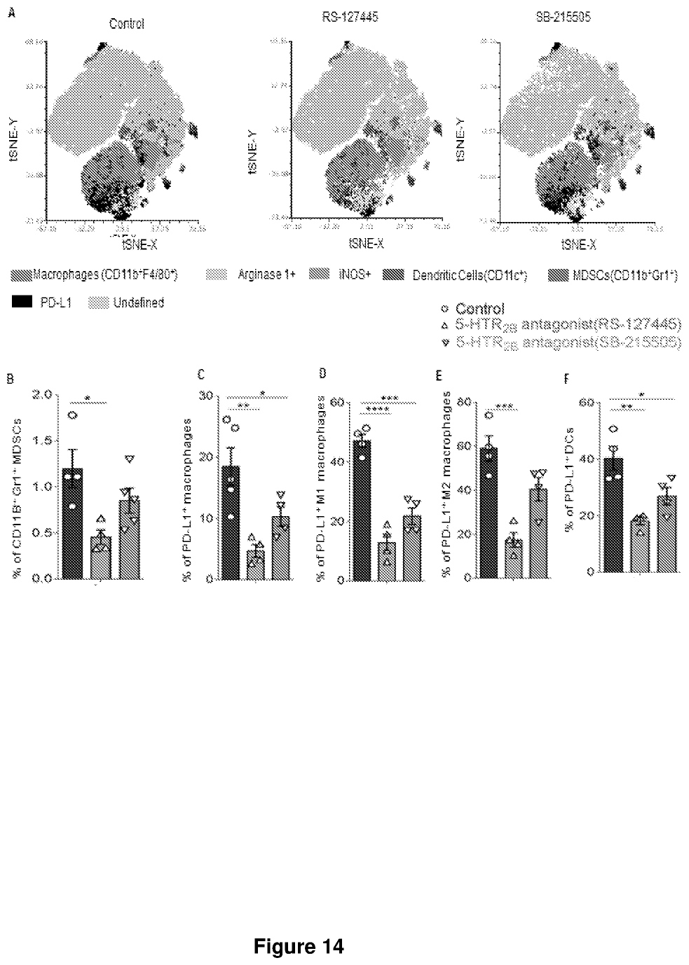

The enhanced tumor progression by serotonin-mediated 5-HTR_{2B} signaling via direct/indirect modulation of immune cells in the TME, in turn, appears to be responsible for the regulation of mood and behavior and contributes to cancer progression. The existence of serotonin in the gut makes it more potent in its role in the modulation of gastrointestinal cancer like colon cancer. As the expression of 5-HTR_{2B} subtypes is much wider in the periphery, its signaling is also very important for the extra-neuronal effect of serotonin. Therefore, 5-HTR_{2B} signaling and serotonin promote tumor progression. To prove the specificity of 5-HTR_{2B} signaling in tumor reduction, pan-5-HTR₂ antagonist ketanserin, 5-HTR_{2A} antagonist ritanserin, and another 5-HTR_{2B} antagonist RS-127445 were tested in the MC38 colon cancer model. It was found that apart from 5-HTR_{2B} antagonists, other antagonists did not prevent tumor growth (FIG. 7A). The 5-HTR_{2B} antagonist RS-127445 proved to be a potent molecule in reducing the tumor burden by modulating the MDSCs and PD-L1⁺ macrophages and DCs in the TME (FIG. 14A-10F).

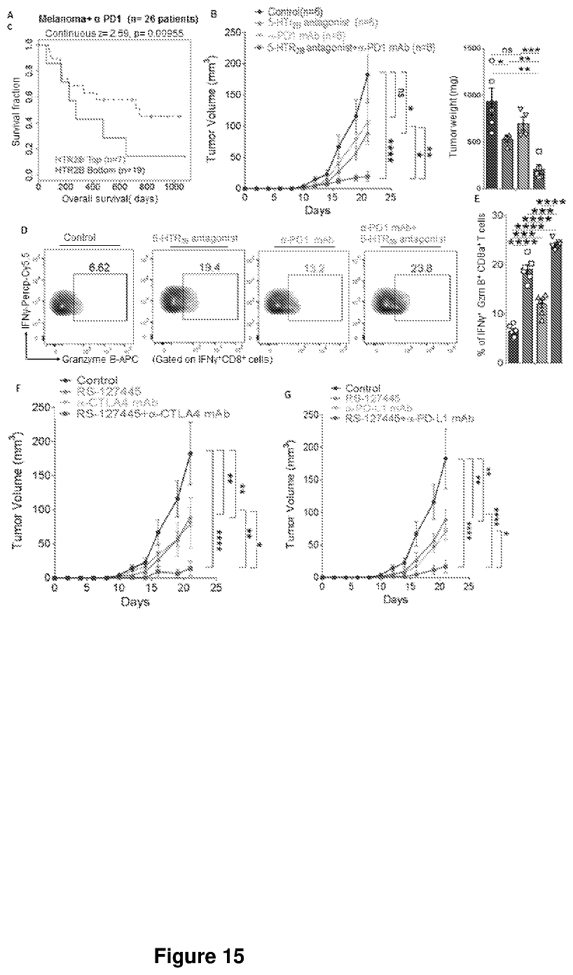

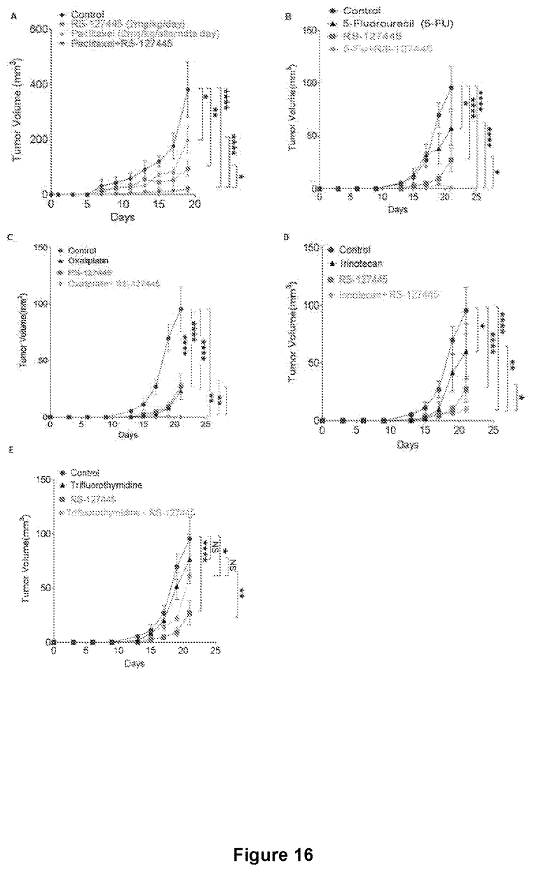

In addition to the use of 5-HTR_{2B} antagonist alone, the combination of 5-HTR_{2B} antagonist with a low dose of FDA-approved immune checkpoint blocker may be selected from the group comprising anti-mouse PD1 monoclonal antibody, clone RMP1-14, anti-mouse PD-L1 monoclonal antibody; clone 10f.9G2, anti-mouse CTLA4 antibody; clone 9H10in tumor-bearing mice, reduced the tumor growth (FIGS. 15B, 15C, 15F, and 15G) and showed an enhanced anti-tumor immune response (FIG. 15D-E). Further, when a 5-HTR_{2B} antagonist with a very low dose of known chemotherapeutic agents selected from a group comprising 5-Fluorouracil, paclitaxel, oxaliplatin, trifluorothymidine, and irinotecan, showed enhanced the efficacy of the drugs such as 5-fluorouracil, paclitaxel, oxaliplatin, and irinotecan but not trifluridine in controlling tumor growth in CRC and breast cancer in mice model (FIG. 16A-E). These results suggested that combining the low dose of chemotherapeutic agents or other immunotherapies strongly modulates the anti-tumor immune response and controls tumor growth.

The serotonin receptor of 5-HTR_{2B} receptor antagonist and immunomodulator selected from the present invention interacts synergistically with one another to provide surprisingly cancer inhibiting properties.

The present invention is illustrated by way of examples. The examples are meant only for illustrative purposes and are not meant to restrict the invention in any manner. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Evaluation of the Efficacy of HTR_{2B} Receptor Antagonists Alone or in Combination with Other Biologics and Chemotherapeutic Drugs of the Present Invention by Different Methods

### Example 1.1: Mouse Model

Six to eight weeks old C57BL/6J (RRID: IMSR_JAX:000664), BALB/c (RRID:IMSR_JAX:000651), NRG (NOD.Cg-Rag1^{tm1Mom} Il2rg^{tm1Wjl}/SzJ), OT-I (C57BL/6-Tg(TcraTcrb)1100Mjb/J), OT-II (B6.Cg-Tg(TcraTcrb)425Cbn/J) male and female mice were used in the tumor studies, were procured from the Jackson Laboratory (Marine, USA) and bred in the experimental animal facility (EAF) of NCCS. All experimental procedures were approved by the Institutional Animal Ethical Committee (IAEC). The animal ethics numbers are EAF/2017/B-256.

### Example 1.2: Cell Lines and Culture Methods

Mice colon adenocarcinoma cell line MC38 and mouse breast cancer cell lines 4T1-Luc2 were received from the NCCS cell repository. MC38 cells were cultured in complete Dulbecco's modified Eagle media (DMEM) supplemented with 10% fetal bovine serum, 2 mM glutamine, 0.1 mM MEM non-essential amino acid, 1 mM sodium pyruvate, 10 mM HEPES, 50 μg/ml gentamycin, and pen/strep. The 4T1-Luc2 cell was cultured in complete RPMI-1640 media (RPMI supplemented with 10% FBS, 1 mM sodium pyruvate, 2 mM glutamine, and pen/strep). All cell lines were maintained at 37° C. in a 5% CO2 incubator.

### Example 1.3: Plasmids, Tetramers Antibodies, and Chemicals

PCI-neo-cOVA was a gift from Maria Castro (Addgene plasmid #25097; http://n2t.net/addgene:25097; RRID:Addgene_25097). H-2K^{b}-ova₂₅₇₋₂₆₄ tetramer (OT-I) and I-A(b)-ova₃₂₉₋₃₃₇ tetramer (OT-II) was received from the NIH tetramer core facility. Rabbit monoclonal Ki67 antibody (Abcam; Catalog #ab16667; 1:100), Alexafluor 488 Rat anti 5-HTR_{2B} antibody (Novus biologicals; catalog #NB100-65037A; 1:100), Rabbit monoclonal anti-TPH1 antibody (Novus biologicals; catalog #NBP2-67580; 1:50), Alexafluor 594 anti-neurofilament H antibody (Biolgend; catalog #801709; 1:100), Goat polyclonal anti-serotonin antibody (Abcam; catalog #ab66047; 1:50), FITC anti-mouse CD31 antibody (Biolegend; catalog #102406; 1:100), FITC anti-mouse F4/80 antibody (Biolegend; catalog #123108), APC anti-mouse iNOS antibody (eBioscience; catalog #17-5920-82), Alexafluor 488 anti-mouse/human arginase 2 antibody (eBiosiences; catalog #53-3697-82), APC/Cy7 anti-mouse CD8 antibody (Biolegend; catalog #100714), Percp/cy5.5 hamster anti-mouse CD69 antibody (BD biosciences; catalog #551113), Percp/cy5.5 anti-mouse IFN 7 antibody (eBioscience; catalog #45-7311 82), Alexafluor 647 anti-mouse/human Granzyme B antibody (Biolegend; catalog #515405), Alexafluor 488 anti-mouse CD44 antibody (Biolegend; catalog #103016), APC anti-mouse CCR7 antibody (eBiosiences; catalog #17-1971-81), PE/cy7 ant-mouse CD4 antibody (Biolegend; catalog #100528), Invivomab rat monoclonal anti-mouse CD8 depletion antibody (BioXcell; cxatalog #BE0117), Biotin anti-mouse IL-10 (Biolegend, catalog #505004), Percp/cy5.5 anti-mouse PD-L1 antibody (Biolegend; catalog #124334), PE/cy7 antimouse/human CDllb antibody (Biolegend; catalog #101216), Biotin anti-mouse Gr1 antibody (Biolegend; catalog #108404), APC/cy7 anti-mouse CD11c antibody (Biolegend; catalog #117324), Invivomab rat monoclonal anti-mouse PD1 antibody (BioXcell; cxatalog #BE0146), Invivomab rat monoclonal anti-mouse PD-L1 antibody (BioXcell; cxatalog #BE0101), Invivomab rat monoclonal anti-mouse CTLA4 antibody (BioXcell; cxatalog #BE0131), Mouse IL-10 ELISA max (Biolegend; catalog #431414), Mouse PD-L1 Duoset ELISA (R& D biosystem; Catalog #DY1019-05), Mouse IL-10 ELISA max (Biolegend; catalog #430904), SB-215505 (Santa cruz biotechnology; catalog #sc-253540), RS-127445 (Sigma; Catalog #R2533), Ketanserin tartarate (Sigma; catalog #S006-10 mg), Ritanserin (Sigma; catslog #R103), Setraline hydrochloride (Abcam; catalog #ab141068).

### Example 1.4: Syngeneic Tumor Model Development

MC38 and MC38-cOVA cells were injected subcutaneously into the right flank of male C57BL/6 or NRG mice (0.5×10⁶ cells/mouse in 150 μl PBS). 4T1-Luc2 cells were injected into the mammary fat pad of female BALB/c mice (0.5×10⁶ cells/mouse in 150 μl PBS). Tumor volume was measured using a Vernier caliper on each alternate day or every third day as soon as the tumor appears. The volume of the tumor was measured using the formula V=L×W²/2, where L=length of the tumor (mm) and W=width of the tumor (mm).

### Example 1.5: TCGA Survival Data Analysis

The correlation between serotonin receptor expression pattern and survival status of subjects with colon adenocarcinoma (COAD) from The Cancer Genome Atlas (TCGA) database was analysed through the oncolnc.org website. The survival status of COAD subjects from TCGA databases showing the highest expression (top 25% segment; n=110) of serotonin receptor subtypes and COAD subjects showing the lowest expression (bottom 25% segment; n=110) of serotonin receptor subtypes were correlated in oncolnc.org, and the Kaplan-Meier plot for every serotonin receptor subtype was plotted.

### Example 1.6: Tumor Immune Dysfunction and Exclusion (TIDE) Computational Method

TIDE analysis was performed as per the details present on the website http://tide.dfci.harvard.edu/. TIDE is used to study the correlation between the tumor-infiltrating CD8 cytotoxic T lymphocytes and overall subject survival in relation to the HTR2B gene expression. For each subject cohort, tumor samples were divided into HTR2B-high (Samples with HTR2B expression one standard deviation above the average) and HTR2B-low (remaining samples) groups, followed by analyzing the association between the CTL levels and surviving outcomes in each group. The CTL functionality was measured based on the average expression of CD8A, CD8B, GZMA, GZMB, and PRF1. Each survival plot presented tumors in two subgroups; “CTL-High” had above average CTL values among all samples, whereas, “CTL-low” had below average CTL values among all samples. A T cell dysfunction score (z score) was calculated for each subject cohort, correlating the HTR2B expression level with the beneficial effect of CTL infiltration on subject survival. A positive z score indicates that the expression of HTR2B is negatively correlated with the beneficial effect of tumor-infiltrating CTL on subject survival.

### Example 1.7: Chromogenic Immunohistochemistry

The human colon adenocarcinoma tissue microarray was used in this study bought from Novus biological (catalog #NBP2-78088) and stained with the antibodies and detected by chromogenic reagent DAB. Rabbit polyclonal anti-human 5-HTR_{2B} antibody (1:100 dilution) and rabbit polyclonal anti-human TPH1 (1:100 dilution) were used as primary antibodies. The goat anti-rabbit HRP tagged secondary IgG (1:200) was used as the secondary antibody. The slides were warmed at 60° C. for 1 hour before staining. The slides were deparaffinized by xylene and dehydrated by graded treatment of alcohol. Then antigen retrieval was performed using antigen-retrieval buffer (Biolegend; catalog #928001; pH 8.0) at sub-boiling temperature for 15 minutes. The intrinsic peroxidase of the tissue was quenched by Peroxidase blocking reagent (Biolegend; catalog #927402). Sections were blocked and permeabilized with 10% goat serum in PBST (0.1 mM PBS; 0.5% Triton-X). The sections were incubated with primary antibody at 4° C. overnight. The slides were washed with PBST (1×PBS, 0.05% Tween 20) and incubated with a secondary antibody for 1 hour at room temperature. Lastly, they are detected with DAB-H₂O₂ reagent and counterstained with haematoxylin, and acquired by brightfield microscopy in Leica DMI6000 fluorescence microscope (Leica Microsystems, Germany).

### Example 1.8: Immunohistochemical Staining

For fluorescence-based immunohistochemical staining, tissues were embedded in OCT tissue freezing media (Fisher Scientific) and stored at −80° C. Tissue sections (7 μM thick) were fixed in chilled acetone for 10 mins, air dried, washed with cold PBST (0.1 mM PBS, 0.05% Tween-20), and blocked with 10% horse serum (Jackson ImmunoResearch, West Grove, P.A.) for 1 hour at room temperature (RT). Sections were washed with PBST and incubated with fluorochrome-tagged primary antibody or purified primary antibody (1:200 dilutions) in 1% animal serum at 4° C. overnight. Sections are washed twice with PBST and stained with secondary antibody (1:800 dilutions) for 1 hour at RT. Then, sections were washed thrice with PBST and fixed with 1% paraformaldehyde, and mounted with DAPI containing aqueous mounting media (Electron Microscopy Sciences, Hatfield, PA). The images were captured using Olympus FV3000 confocal microscope (Waltham, Massachusetts), and the images were analysed using Cellsens software (Olympus; Waltham, Massachusetts).

### Example 1.9: RNA Isolation and Semi-Quantitative PCR

Total RNA was isolated from purified cells using TRIZOL reagent (Invitrogen). RNA was measured, and DNA contamination was removed using DNAse I. cDNA was prepared using Omniscript RT kit (Qiagen), and qRT-PCR was performed using a specific forward and reverse primer mix and a universal SYBR green reverse transcriptase master mix (Biorad; catalog #1725122) in CFX96 Touch deep well Real-Time PCR system (Biorad).

### Example 1.10: Preparation of Single-Cell Suspension and Cell Staining for Flow Cytometry

Tumors were excised, manually disrupted into small pieces using fine forceps and scissors, and resuspended in serum-free DMEM media containing collagenase type I (0.1 mg/ml), collagenase IV (0.1 mg/ml), hyaluronidase (0.06 mg/ml), DNase I (0.02 mg/ml) and soybean trypsin inhibitor (0.1 mg/ml) and incubated for 30-60 minutes at 37° C. in a shaker incubator. The single-cell suspension was prepared by passing through a 70 μM pore-size cell strainer. Then cells were washed with PBS and stained with fluorochrome-tagged primary antibodies. Single-cell suspension of spleen and lymph nodes was prepared by mechanical disruption of the tissues, and the cell suspension was passed through a 70 μM pore size cell strainer. RBCs were removed using ACK lysis buffer, washed with RPMI medium, and stained using specific antibodies.

Cells were incubated with fluorochrome-tagged primary antibodies for 1 hour on ice. Then, washed once with PBS, and cells were either fixed with 0.5% paraformaldehyde or proceeded for intracellular staining. For intracellular staining, cells were fixed with 1×FoxP3 fixation buffer (Biolegend) for 45 minutes on ice, followed by washing with 1× permeabilization buffer (Biolegend) and incubated with permeabilization buffer for 30 mins on ice. Cells were then incubated with fluorochrome-tagged specific antibodies in permeabilization buffer for 1 hour, washed with PBS, and fixed with 0.5% paraformaldehyde. Finally, the cells were acquired in the FACS Canto II instrument (B.D. Bioscience), and the data were analysed using FlowJo software (TreeStar).

### Example 1.11: Cell Cycle Analysis and MTT Assay

MC38 cells were seeded in 6 well plates at a density of 5×10⁴ cells/well. Cells were allowed to accommodate in the environment for 24 hours. The treatment with 5-HTR2B agonist (BW-723C86), Serotonin, and 5-HTR2B antagonist (RS-127445) was performed at different time points. After that, the cell was harvested by treating them with 0.25% Trypsin-EDTA solution at 37° C., washed with PBS, and fixed with 75% ethanol for 1 hour. Cells were washed with PBS and stained with propidium iodide and acquired in FACS Canto II instrument (B.D. Biosciences)

MC38 cells were cultured at a density of 0.5×10⁴ cells/well in 96 well plates (flat bottom) and were allowed to adhere for 24 hours. Then, 5-HTR2B agonist (BW-723C86), Serotonin, and 5-HTR2B antagonist (RS-127445) for 48 and 72 hours in a reduced serum medium (2% FBS containing DMEM). Then, the media was removed, washed once with PBS, and incubated with MTT reagent for 3 hours at 37° C. Then, MTT crystals were dissolved with DMSO, and the reading was taken at 590 nm in an ELISA reader (Thermofisher).

### Example 1.12: ELISA

Universal competitive serotonin ELISA kit was purchased from Novus biological (Catalog #NBP2-68127), and ELISA was performed according to the manufacturer's protocol. Mouse PD-L1 DuoSet ELISA kit was procured from the R&D System (Catalog #DY1019-05), and the ELISA was performed according to the manufacturer's guidelines.

### Example 1.13: Statistical Analysi

Unpaired two-tailed Student's t-test was used to compare two independent groups. For comparing more than two independent groups, ANOVA with multiple comparison tests was used. A p-value of less than 0.05 was considered statistically significant. All statistical analyses were performed using GraphPad Prism 6 software (GraphPad Software, San Diego, CA).

### Example 2: Overexpression of 5-HTR_{2B} in Human Colon Adenocarcinoma (COAD) Correlates with Poor Survival of Subjects

To understand the impact of the serotonergic receptor in the tumor, the RNAseq data of colon adenocarcinoma (COAD) subjects from The Cancer Genome Atlas (TCGA) database was analyzed. These data correlate the expression of different serotonin receptor subtypes in the cancer tissues and the 10-year survival rate of those subjects. The correlation of various other cancer subject's survival (Bladder urothelial carcinoma, Cervical squamous cell carcinoma, Glioblastoma, Kidney renal clear cell carcinoma, Breast invasive adenocarcinoma, Esophageal carcinoma, Head and Neck squamous cell carcinoma, Kidney renal papillary cell carcinoma, Acute myeloid leukemia, Liver Hepatocellular Carcinoma, Lung squamous cell carcinoma, Pancreatic adenocarcinoma, Brain lower grade Glioma, Lung adenocarcinoma, Ovarian serous cystadenocarcinoma, Rectal adenocarcinoma, Sarcoma, skin Cutaneous Melanoma, and Uterine Corpus endometrial carcinoma) in relation with 5-HTR_{2B} expression, and none except acute myeloid leukemia and stomach adenocarcinoma cancers from different tissues showed any significant correlations (FIG. 1). Among all, colon adenocarcinoma cancer showed a strong negative correlation between 5-HTR_{2B} expression and the survival of COAD subjects (FIG. 2A). Further, using a tissue array of human COAD subjects we have observed that higher 5-HTR_{2B} expression in the colon tumor also correlates with the high number of Ki67⁺ proliferating cancer cells in the colon tumor (FIG. 2B). The expression of serotonin synthesizing enzyme TPH1 in tumor microarray showed that human COAD tissues had enhanced expression of 5-HTR_{2B} (FIG. 2B) and TPH1 (FIG. 2C) in the tumor as well as in the infiltrating cells in the lamina propria as compared to control tissues. To check whether enhanced TPH1 expression correlates with the expression of serotonin in the tumor, the tumor microarray tissue was stained with anti-serotonin and anti-neurofilament H antibodies (to stain the neurons) (FIG. 2D). It was observed that in tumor conditions both the neuronal and non-neuronal sources of serotonin are enhanced. This enhanced expression of 5-HTR_{2B} and serotonin in the tumor microenvironment indicates altered serotonin physiology in the colon tumor.

### Example 3: Colon Tumor Enhances the Overall Serotonin Level in Serum and the Tumor Over Time

As the serotonergic system is altered in the colon tumor in humans, to observe the effect of the serotonergic system in the colon tumor, colon tumor in mice was developed by subcutaneously injecting MC38 cells in the C57BL/6J mice. To check the intratumoral expression of serotonin, TPH1, and 5-HTR_{2B} in the course of tumor growth, the tumor was harvested at a seven-day interval after days 7, 14, and 21 days of tumor inoculation and stained the tumor sections with anti-serotonin, anti-TPH1, and anti-5-HTR_{2B} antibody. An enhanced expression of 5-HTR_{2B} within the tumor in the later stages was observed (FIG. 3B). However, the expression of serotonin and TPH1 remained constant throughout tumor growth (FIG. 3B). The serum serotonin level was tracked in the MC38 tumor-bearing mice from the above experiments and compared with the naïve mice kept in a similar condition (FIG. 3C). It was found that the serum serotonin level was significantly higher in the MC38 tumor-bearing mice than in the naïve mice (FIG. 3C). Altogether, these data suggest that the MC38 tumor contributes to higher levels of serotonin in those mice and as this tumor has higher 5-HTR_{2B} expression during tumor growth; they can respond to the enhanced serotonin levels quite efficiently.

### Example 4: Mouse Colon Adenocarcinoma (MC38) and Show Expression of 5-HTRs and TPH1, DDC, and MAO-A

To understand the detailed role of 5-HTRs in colon adenocarcinoma, mouse colon adenocarcinoma cell line MC38 was used to monitor the expression of 5-HTRs and other serotonergic systems. Semiquantitative PCR analysis of MC38 cDNA showed a variable expression of several 5-HTR subtypes (FIG. 4A-B). However, 5-HTR_{1B}, 5-HTR_{2B}, and 5-HTR₇ expressions were higher in these cells with no indication of 5-HTR₄ and 5-HTR₆ expressions (FIG. 4A). MC38 cells also showed higher expression of serotonin synthesizing enzyme TPH1 (FIG. 4C), suggesting that these tumor cells express 5-HTRs, and are capable of producing serotonin. The qRT-PCR data from this cell line also showed variable expression of serotonin receptors (FIG. 4D) and these cells not only express TPH1 but also express DOPA decarboxylase (a downstream enzyme of TPH1 in the serotonin synthesis pathway) and Monoamine oxidase-A (the enzyme responsible for the degradation of serotonin and many other monoamine compounds) (FIG. 4E). These data suggest that MC38 colon adenocarcinoma cell lines can synthesize, degrade, and respond to serotonin signalling.

### Example 5: Different Immune Cell Subsets Show the Expression of Serotonin Receptors and Enzymes of Serotonin Synthesis and Degradation Pathways

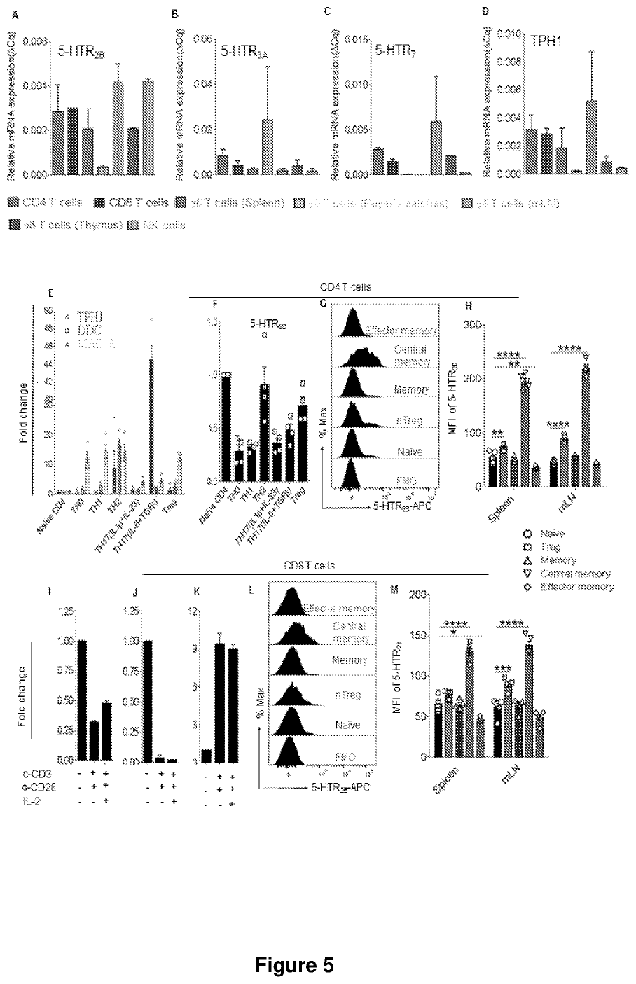

Not only tumor cells, but the immune cells are also the major component of the tumor and they contribute to either tumor regression or progression significantly. These immune cells respond to serotonergic signalling and can contribute to tumor progression. To confirm this hypothesis, the expression of various serotonin receptor subtypes and TPH1 was checked in different immune cell subsets such as CD4 T cell, CD8 T cell, γδ T cells, and NK cells (FIG. 5A-D). All of these show the variable expression of 5-HTRs (5-HTR_{2B}, 5-HTR₇, and 5-HTR_{3A}). Not only that they also express TPH1, the rate-limiting enzyme for serotonin biosynthesis. These data suggest that subsets of the innate and adaptive immune cells can also produce serotonin. As they also express different serotonin receptor subtypes to varying degree and they can also respond to serotonin-mediated signalling through autocrine, paracrine, or a juxtracrine manner. As the CD4 and CD8 T cells are the main effector cells in the tumor, the expression of these receptors was observed in the various subtypes of these cells. The expression of 5-HTR_{2B} and TPH1, DDC, and MAO-A in vitro differentiated mice CD4 T cells into Th0, Th1, Th2, pathogenic and non-pathogenic Th17 and Treg cells using cocktails of various cytokines, and the expression were also identified (FIG. 5E-F). It was found that among different subtypes of in vitro differentiated CD4 T cells, anti-inflammatory Th2, Tregs and Th0 have higher expression of 5-HTR_{2B} (FIG. 5F). The said cells have the reputation of being the tumor-promoting cells in vivo and they also show variable expression of TPH1, DDC, and MAO-A (FIG. 5E). To check the expression of 5-HTR_{2B} on the various subtypes of CD4 T cells in vivo, various subtypes of CD4 T cells from the spleen and mLN of naïve mice were stained using different markers along with 5-HTR_{2B} and analysed by flow cytometry. It was found that naïve, natural Tregs and central memory CD4 T cells have higher expression of 5-HTR_{2B} compared to other subtypes (FIG. 5G-H). The expression of 5-HTR_{2B} and TPH1, DDC, and MAO-A in vitro stimulated CD8 T cells were studied and it was found that CD8 T cells stimulated in vitro with α-CD3, α-CD28, and IL-2 reduce the expression of 5-HTR_{2B} and TPH1 but the enhanced expression of serotonin degrading enzyme MAO-A (FIG. 5I-K). To check the expression of 5-HTR_{2B} on the various subtypes of CD4 T cells in vivo, various subtypes of CD8 T cells from the spleen and mLN of naïve mice using different markers along with 5-HTR_{2B} were stained and analysed by flow cytometry. It was observed that naïve, natural Tregs and central memory CD8 T cells have higher expression of 5-HTR_{2B} compared to other subtypes (FIG. 5L-5M). These data suggest not only cancer cells, but also various subtypes of immune cells also express different components of the serotonergic system.

5-HTR_{2B} is expressed widely in dendritic cells and macrophages and its expression is upregulated in M2 macrophages during polarization. Previous reports are suggesting that 5-HTR_{2B} signalling promotes the polarization of M2 macrophages. Similarly in DCs, an earlier report suggests the expression of 5-HTR_{2B} and TPH1 in various DC subsets and serotonin signalling through 5-HTR_{2B} indeed promotes the formation of regulatory DC subsets which ultimately inhibits the formation of pro-inflammatory Th1 cell polarization. The expression of 5-HTR_{2B} is also enhanced in DCs and macrophages within the tumor than in non-draining lymph nodes such as the spleen in mice with colorectal cancer.

### Example 6: Agonizing the 5-HTR_{2B} Promotes Colon Cancer Cell Proliferation and Viability

As the expression of serotonergic signaling is also extended in the immune components of tumors whether 5-HTR_{2B} signaling in tumors directly affects the cancer cells or immune cells or indirectly modulates immune cells via cancer cells needed to be tested. To test this, the effect of serotonin-5-HTR_{2B} signaling on the proliferation and viability of the MC38 mouse colon cancer cell line was tested. MC38 cells were treated with increasing doses of 5-HTR_{2B} agonist BW-723C86 or serotonin. Following 48-hour stimulation, the 5-HTR_{2B} agonism enhanced the cell division, which was evident from the increased percentage of cells in the S phase of the cell cycle. In contrast, the antagonism of this receptor reduced the percentage of MC38 cells in the S phase of the cell cycle (FIG. 6A). The graded dose of 5-HTR_{2B} agonist BW-723C86 and serotonin also enhanced the viability of MC38 cells in reduced serum media following 48 hours of stimulation (FIG. 6B). The effect of 5-HTR_{2B} signaling on the angiogenesis in the colon tumor was checked through the development of colon tumor in mice with an MC38 cell line and then treating them with 5-HTR_{2B} antagonist RS-127445. After 20 days of tumor inoculation, the tumor was harvested and IHC was performed by staining it with an anti-CD31 antibody to observe the numbers of CD31⁺ micro-blood vessels in the tumor. It was observed that 5-HTR_{2B} antagonism reduced the number of micro-blood vessels/mm² in the tumor area (FIG. 6C). Together, these results showed that 5-HTR_{2B} signaling promotes the proliferation and viability of colon cancer cells and promotes angiogenesis in the tumor bed.

### Example 7: Antagonizing the 5-HTR_{2B} in Mice Reduces the Colon Cancer, Breast Cancer and Melanoma Tumor Burden in Mice

An MC38 colon tumor was developed in mice as above stated method and the mice were treated with two different 5-HTR_{2B} antagonists (SB-215505 and RS-127445), a pan-5-HTR₂ blocker (Ketanserin), and a 5-HTR_{2A} antagonist (Ritanserin) (FIG. 7A). It was observed that both the 5-HTR_{2B} antagonists efficiently control the tumor growth. Ritanserin also controls tumor growth to some extent but Ketaserin completely fails to control tumor growth. The blocker of the 5-HTR₇ was tested on the MC38 tumor growth and they failed to produce any effective results (FIG. 7B). The effect of enhanced total free serotonin levels in the blood on colon cancer progression was observed by treating them with serotonin reuptake inhibitor (SSRI) sertraline (FIG. 7C). These SSRI drugs are used to treat many depressions like symptoms as they enhance total available serotonin levels in the blood by preventing the reuptake of serotonin in the storage cells. It was observed that high free serotonin in the blood promotes MC38 tumor growth (FIG. 7C). The 5-HTR_{2B} antagonists are the most efficient in controlling MC38 tumor growth in mice, the effect of 5-HTR_{2B} antagonists was tested. In the beginning, a dose-response curve of 5-HTR_{2B} antagonist SB-215505 was created by using an array of doses ranging from 0.5 mg/kg/day to 4 mg/kg/day. It was observed that 4 mg/kg/day is the most efficient dose in reducing MC38 tumor growth (FIG. 7D). The impact of agonism of the 5-HTR_{2B} receptor on MC38 tumor growth was observed and it was found that 5-HTR_{2B} agonist BW-723C86 significantly promotes MC38 tumor growth (FIG. 7E). The effect of 5-HTR_{2B} antagonist cariprazine on colon tumor growth was also observed. Cariprazine is an FDA-approved compound for treating schizophrenia-like symptoms in subjects (FIG. 7F). Apart from the 5-HTR_{2B} antagonist property, cariprazine also acts as a strong agonist of dopamine receptor subtypes such as D2 and D3. Oral supplement of cariprazine showed a significant reduction in MC38 tumor growth in vivo (FIG. 7F). The mice breast cancer model was tested by using 4T1-Luc2 mice breast cancer cell line and it was found to have similar results where 5-HTR_{2B} antagonist reduced tumor burden (FIG. 7G). We have also tested the efficacy of 5-HTR_{2B} antagonist treatment of melanoma growth in mice. For that, we have developed mice melanoma model by subcutaneously injecting B16F10 cells in the left flank of C57BL/6J mice and treated one group of mice with 5-HTR_{2B} antagonist SB-215505. Similarly, like colon and breast cancer, we have also found reduced tumor burden in the mice melanoma model (FIG. 7H). Further, cellular analysis of the tumor-infiltrating immune cells in the said mice showed a decreased percentage of total F4/80⁺ macrophages and arginase 1⁺ (Arg1⁺) M2 macrophages (anti-inflammatory) in the tumor following treatment with 5-HTR_{2B} antagonist (FIGS. 7I and 7K). However, this treatment did not induce a significant change in the percentage of iNOS⁺ M1 macrophages (inflammatory macrophages) in the tumor microenvironment (FIG. 7J). Immunohistochemical staining of the tumors from control and 5-HTR_{2B} antagonist treated mice has shown decreased localization of M2 macrophage (Arginase 1⁺F4/80⁺ cells) and CD11c⁺ DCs in the tumor microenvironment (TME), but there were not many changes in the localization of M1 macrophage in the TME following with treatment (FIG. 7L). Taken together, all data suggests that 5-HTR_{2B} signaling promotes colon tumor growth, and blocking that signaling suppresses tumor growth in vivo.

### Example 8: Adoptively Transferred 5-HTR_{2B} Antagonist-Treated Immune Cells Reduced Tumor Growth in Immunocompromised Mice

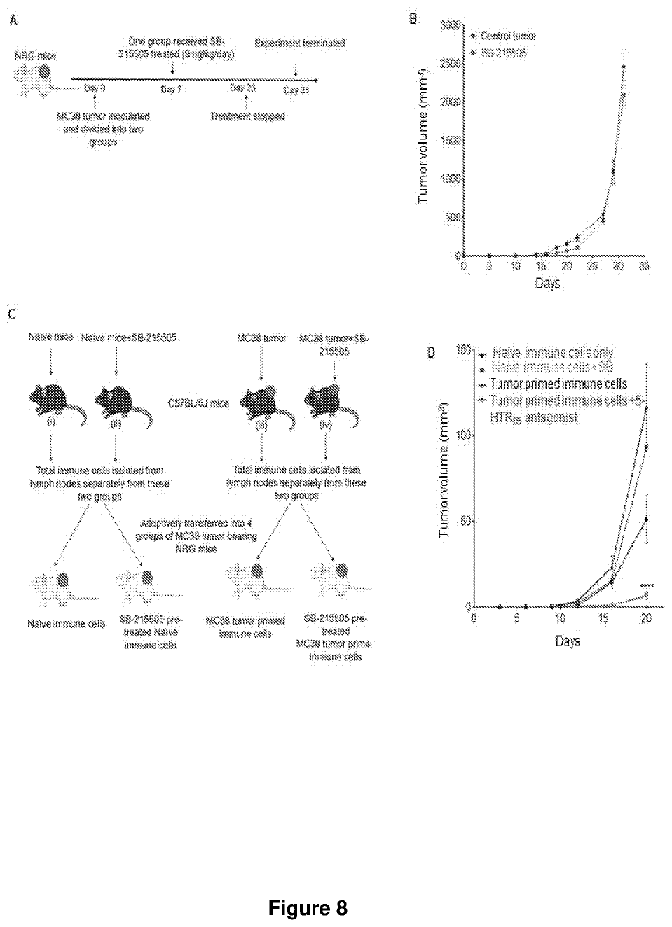

An immuno-compromised NRG (NOD Rag1^{-/-} IIL2rγ^{-/-}) mice that lack T cells, B cells, and NK cells were taken to study the working mechanism of the 5-HTR_{2B} antagonist. NRG mice were given subcutaneous injections of MC38 cells. After 7 days of tumor inoculation, mice were either given 5-HTR_{2B} antagonist SB-215505 (4 mg/kg/day) or left untreated as control (FIG. 8A). After 23 days of MC38 injection, the supply of 5-HTR_{2B} antagonist was stopped in the mice, and tumor growth was monitored. It was observed, if mice were treated with SB-215505, tumor growth was suppressed to some extent (FIG. 8B) but not as much as in the wild-type mice (FIG. 7E). These data suggest that the presence of adaptive immune cells and NK cells is required for the anti-tumor activity of 5-HTR_{2B} antagonist in mice.

To observe whether immune cells have any impact on the anti-tumor activity of the 5-HTR_{2B} antagonist. MC38 tumor-bearing mice were treated with a 5-HTR_{2B} antagonist and after 10 days of such treatment, lymph node cells from these mice were harvested and adoptively transferred into groups of fresh MC38 tumor-bearing NRG mice and the growth of tumors were monitored. Adoptive transfer of naïve mice lymph node cells was used as control as shown in FIG. 8C. The results showed that adoptive transfer of only 5-HTR_{2B} antagonist-treated lymph node cells suppressed the growth of tumors in NRG mice (FIG. 8D), which was not seen with naïve lymph node cells or cells from only MC38 tumor-bearing wild-type mice (FIG. 8D). This data indicated that 5-HTR_{2B} antagonists suppress colon tumor growth by affecting immune cells not the cancer cell in vivo.

### Example 9-5-HTR_{2B} Antagonist Suppresses Tumor Growth by Affecting CytotoXIc T Cell Response in Tumor

As, we have observed 5-HTR_{2B} signaling affects innate immune cells and cancer cells in such a way which can promote overall tumor progression, its effect on the main effector cells against tumors such as CD4 and CD8 needed to be studied. The CD8 (CTLs) in the tumors was studied through TIDE (Tumor Immune Dysfunction and Exclusion) database from Harvard University. This database can be used to visualize the impact of any gene expression on the cytotoxic T cell (CTLs) response in the tumor. The impact of 5-HTR_{2B} expression on the CTLs response in colon tumors was studied (FIG. 9A). It was found that when there is a higher expression of 5-HTR_{2B} in the colon tumors, the CTLs are not dysfunctional, i.e, even higher CTL counts in the tumor failed to control tumor progress as it is evident from no change in overall survival of colon cancer subject (left side; FIG. 9A). Conversely, when there is a lower expression of 5-HTR_{2B} in the tumor high CTL count is associated with better overall survival of colon cancer subjects (right side; FIG. 9A). This data suggests higher 5-HTR_{2B} expression in tumors is associated with dysfunctional CTLs in the tumor and the worst prognosis of the disease. To observe its actual relevance in colon cancer progression, a mouse with the MC38 colon cancer model was used and treated with a 5-HTR_{2B} antagonist and checked for overall CTL profiles in the tumor. It was found that 5-HTR_{2B} antagonism enhances CD69⁺ activated CD8 T cell population in tumors (FIG. 9B-C). Not only that, but this antagonism also enhanced the total IFNγ⁺ and IFNγ⁺ granzyme B⁺ CD8 T cell percentage in the tumor (FIG. 9D-K). This IFNγ⁺ granzyme B⁺ CD8 T cell is the actual effector CTLs that performs major anti-tumor functions in the tumor. The impact of the antagonism on the memory phenotypes of CD8 T cells was studied and it was observed that 5-HTR_{2B} antagonism also promotes CD44⁺CCR7⁺ central and CD44⁺ CCR7⁻ effector memory CD8 T cells (FIG. 9L-N).

Further, to confirm that CD8 T cell response is the main factor that affects the anti-tumor response of 5-HTR_{2B} antagonism, the MC38 tumor-bearing C57 mice were depleted the total CD8 T cells by using anti-CD8a monoclonal antibody and treated them with 5-HTR_{2B} antagonist (FIG. 10A-B). It was found that depletion of CD8 T cells completely removed the anti-tumor response of the 5-HTR_{2B} antagonist (FIG. 10A). An adoptive transfer experiment was performed to validate the said data, where the treated MC38 tumor-bearing C57BL/6J mice with 5-HTR_{2B} antagonist for 10 days, and then the tumor was harvested and total intra-tumoral CD8 T cells were sorted from 5-HTR_{2B} antagonist treated and untreated MC38 tumor-bearing mice and adoptively transferred them into already MC38 tumor-bearing NRG mice (FIG. 10C). The effect of the antagonism on the intra-tumoral CD4 population was observed and it was found that, though the antagonism does not have any impact on IFNγ⁺ pro-inflammatory CD4 T cell population (FIG. 10D-E), the 5-HTR_{2B} antagonism reduced the percentage of IL-10⁺ regulatory CD4 T cell subsets in the tumor (FIG. 10F-G). Not only that, but 5-HTR_{2B} antagonism also reduced systemic serum IL-10 levels (FIG. 10H) and enhanced serum TNFα levels in MC38 tumor-bearing mice (FIG. 10I). Altogether, these data suggest that 5-HTR_{2B} antagonism enhances the cytotoxic T cell response and reduces regulatory CD4 T cell response in colon cancer which subsequently helps in cancer reduction.

### Example 10—Antagonizing 5-HTR_{2B} Enhances Antigen-Specific CTL Response in Tumor

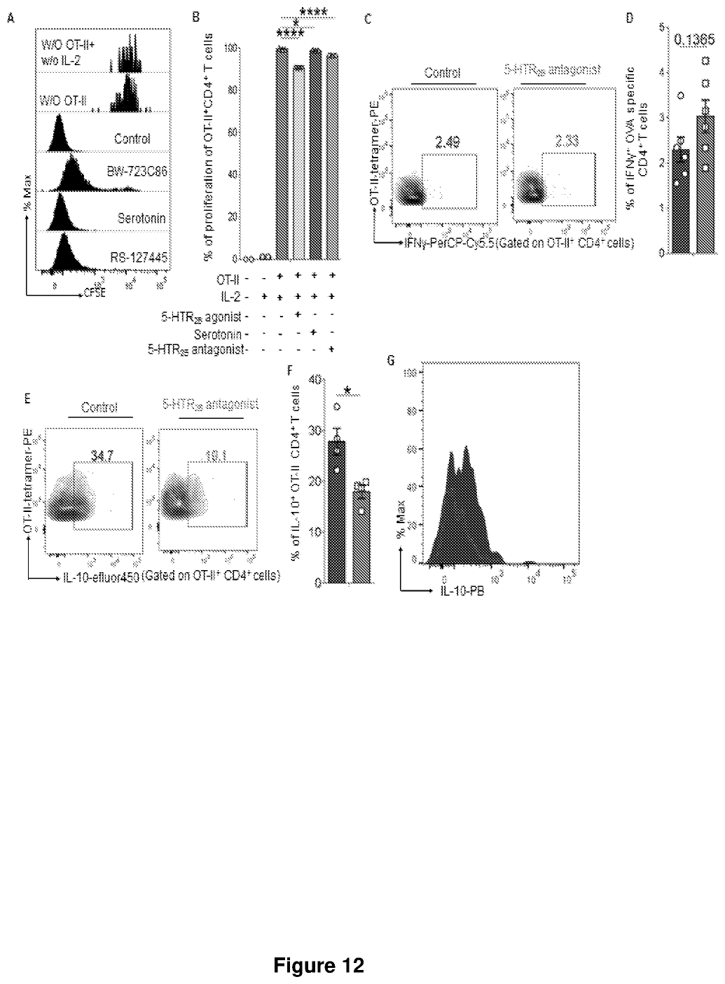

A simple adoptive transfer experiment was performed to study the enhancement of CTL response in general or antigen-specific. Three groups of naïve C57BL/6J (n=3) mice were taken and among them, 2 groups of mice were inoculated with MC38 cells; among them, one group received treatment with a 5-HTR_{2B} antagonist. After 10 days of such treatment, tumor-draining lymph nodes from naïve, MC38−control, and MC38-5-HTR_{2B} antagonist treated mice were collected and the single cell suspension was prepared separately. Then those cells were adoptively transferred separately into 3 groups of NRG mice bearing MC38 tumor on one flank and B16F10 melanoma tumor on the other flank (FIG. 11A). It was observed that 5-HTR_{2B} antagonist primed total immune cells were able to control MC38 tumor growth in one flank but failed to control B16F10 tumor growth on the other flank (FIG. 11B). This data indicates that the 5-HTR_{2B} antagonist may be enhanced the MC38 tumor-specific CTL response. But, to confirm this phenomenon further, an MC38-cOVA cell line was developed by transfecting the MC38 cell line with a pCl-neo-cOVA plasmid (Addgene) (FIG. 11C). This plasmid has a construct that expresses chick ovalbumin protein that lacks a c-terminal secretory segment. The said chicken ovalbumin (OVA) protein is highly immunogenic in mice and the cytoplasmic localization of this protein allows it to be processed and presented through MHC class I molecule and activate CD4 T cells and CD8 T cells, preferentially CD8 T cells. To examine OVA-specific CTL response, this cell was injected into C57BL/6J mice and treated with a 5-HTR_{2B} antagonist. After a few days, intra-tumoral CD8 T cells were harvested and checked for OVA-specific expansion of the CD8 T cells by stimulating them in vitro with total OVA protein (FIG. 11D-E). It was found that in the 5-HTR_{2B} antagonist treated condition intra-tumoral CD8 T cells proliferated at a better rate in the presence of OVA in vitro (FIG. 11D-E). To further check the effect on antigen-specific CTLS and CD4 response MHC class I(H-2K^{b})-ova (SIINFEKL) tetramer (OT-I tetramer) was used to specifically detect OVA-specific CD8 T cells in vivo. It was found that, though 5-HTR_{2B} antagonist did not enhance the overall percentage of OVA-specific CD8 T cell in the tumor (FIG. 11F-G), it enhanced CD69⁺ activated, IFNγ⁺ granzyme B⁺ effector OVA-specific CTLs percentage in the tumor (FIG. 11H-M). OVA-specific CD4 T cell response with 5-HTR_{2B} antagonist was also studied on OT-II mice which has a transgenic CD4 T cell receptor that only recognizes Ova₃₂₃₋₃₃₉ peptide of ovalbumin antigen. The CFSE-tagged total lymph node cells from OT-I mice were cultured in the presence or absence of OT-II peptide (Ova₃₂₃₋₃₃₉) 5-HTR_{2B} agonist (BW-723C86), serotonin, and 5-HTR_{2B} antagonist (RS-127445) as indicated in the figure for 5 days and the proliferation was measured (FIG. 12A-B). It was observed that the 5-HTR_{2B} agonist significantly reduced OT-II peptide-specific proliferation of OT-II CD4 T cells (FIG. 12A-B). The effect of Ova-specific CD4 T cell response was checked, by injecting OT-IH mice with MC38-cOVA cells and treating them with a 5-HTR_{2B} antagonist. It was found that the 5-HTR_{2B} antagonist increased Ova-specific IFNγ⁺ CD4 T cells (FIG. 12C-D) in the tumor and subsequently reduced Ova-specific IL-10⁺ CD4 T cells in the tumor (FIG. 12E-G). Taken together, the data suggest 5-HTR_{2B} antagonism enhances antigen-specific effector CTL response and tumor and concurrently reduces antigen-specific regulatory CD4 T cell response in colon cancer.

### Example 11: Antagonizing the 5-HTR_{2B} in Colon Cancer Promotes the Expression of PD-L1 Molecules

The data showed the inhibition of tumor growth via enhancement of effector response of CTLs by 5-HTR_{2B} antagonism, further experiments were done to know whether this mechanism occurs directly through the action of 5-HTR_{2B} signaling on CD4 or CD8 T cells or it indirectly modulates the TME in such a way that renders it more suppressive to CD4 and CD8 functions. To understand it, investigations were done on whether 5-HTR_{2B} signaling also affects the surface expression of molecules that alters inflammatory and cytotoxic immune cells. Among different molecules, PD-L1 is of particular interest as it is an immune checkpoint ligand that affects cytotoxic T-cell responses toward cancer. The data showed that stimulation of 5-HTR_{2B} with a specific agonist increased the expression of PD-L1 in the colon cancer cells (FIG. 13A-E). Serotonin which has a short life in vitro did not significantly alter the PD-L1 expression, whereas antagonizing the 5-HTR_{2B} reduced the expression of PD-L1 (FIG. 13A-E). Furthermore, treating MC38 tumor-bearing mice with 5-HTR_{2B} agonist BW-723C86 increased the circulating free PD-L1 in the serum, which was diminished upon antagonizing the 5-HTR_{2B} with RS-127445. (FIG. 13F). These data suggest serotonin modulates the PD-L1 level through 5-HTR_{2B} signaling.

### Example 12: Effect of Pan 5-HTR₂ Antagonists or a Specific 5-HTR_{2B} Antagonist on Tumor Growth

Not only the cancer cells, innate immune cells in the TME such as macrophages, DCs, or mononuclear-derived suppressive cells (MDSCs) also are the major cells that express PD-L1 and contribute to CTL suppression. So the PD-L1 expression pattern was checked on above mentions innate cells in TME of MC38 colon tumor following treatment with both 5-HTR_{2B} antagonist SB-215505 and RS-127445. RS-127445 showed an even more potent effect in controlling tumor growth compared to SB-215505 at an equivalent dose. RS-127445 also better modulated the immune response to more effector phenotype in the tumor microenvironment. In the RS-127445 treated tumor there is much reduced percentage of total CD11b⁺Gr1⁺ MDSCs, PD-L1⁺ total macrophages, PD-L1⁺iNOS⁺ M1 macrophages, PD-L1⁺ arginase1⁺ M2 macrophages and PD-L1⁺ CD11c⁺ DCs as compared to control and SB-215505 treated tumor (FIG. 14A-F). Apart from that, this RS-127445 is also reducing the total CD11c⁺ DCs in the TME. But from the tSNE (FIG. 14A) plot it can be observed that 5-HTR_{2B} antagonism reduced the CD11b⁺ PD-L1⁺ CD11c⁺ DCs in the TME; which are recognized as more regulatory DCs, whereas, keeping other DC islands intact. Though further study is needed to dissect the effect of this antagonism on DC-mediated T cell activation. These overall effects may be because RS-127445 is more specific to the 5-HTR_{2B} receptor subtype (almost 1000 times more specific), and it has a higher bioavailability than other 5-HTR_{2B} antagonists. Altogether, this data indicates among different subtypes of 5-HTR₂ receptor subtypes, antagonism of the 5-HTR_{2B} subtype is more potent in reducing tumor growth in mice. Though there may be a difference in the degree of inhibition, 5-HTR antagonists effectively control colon cancer in mice.

### Example 13: Combining a Very Low Dose of Anti-PD1 Antibody with a 5-HTR_{2B} Antagonist Gives a Better Anti-Tumor Immune Response and Controls Tumor Growth

5-HTR_{2B} antagonisms have an anti-tumor effect by reducing PD-L1 expression on the cancer cells and tumor-infiltrating immune cells; this antagonism may also reduce the intrinsic resistance of the tumor to the anti-PD1 and α-PD-L1 immune checkpoint blockade therapy. As this antagonist therapy enhanced CTL response, so it may also remove the resistance to anti-CTLA4 therapy. To test this hypothesis, 5-HTR_{2B} antagonists were used in combination with anti-PD1, anti-PD-L1 and anti-CTLA4 monoclonal antibody therapy. 5-HTR_{2B} antagonists in combination with anti-PD1, anti-PD-L1 and anti-CTLA4 antibodies drastically reduced tumor growth in MC38 colon cancer compared to control or only anti-PD1, anti-PD-L1 and anti-CTLA4 antibodies (FIG. 15A, 15F-G). It was also observed that 5-HTR_{2B} antagonism in combination with anti-PD1 therapy significantly enhanced the IFN-γ⁺ granzyme B⁺ effector CTL population in the tumor (FIG. 15D-E). These data suggest that due to enhanced CTL response and reduced PD-L1 expression by 5-HTR_{2B} antagonism combination of 5-HTR_{2B} antagonists with immune checkpoint blockers The immune checkpoint blockers may be selected from the group comprising anti-mouse PD1 monoclonal antibody, clone RMP1-14, anti-mouse PD-L1 monoclonal antibody; clone 10f.9G2, anti-mouse CTLA4 antibody; clone 9H10, most preferably anti-PD1, anti-PD-L1 and anti-CTLA4 even low doses produce much efficient anti-tumor response in vivo.

### Example 14: Combining a Low Dose of 5-HTR_{2B} Antagonist with Clinically Approved Chemotherapeutic Drugs Gives a Better Anti-Tumor Immune Response and Controls Tumor Growth

The combinations of 5-HTR_{2B} antagonists with immune checkpoint blockers selected from the group comprising anti-mouse PD1 monoclonal antibody, clone RMP1-14, anti-mouse PD-L1 monoclonal antibody; clone 10f.9G2, anti-mouse CTLA4 antibody; clone 9H10 produced much better efficacy. Combinations of 5-HTR_{2B} antagonists were further tested with clinically approved different types of chemotherapeutic agents producing an altered response. Different types of common chemotherapeutic agents can be selected from a group comprising Paclitaxel (Taxol), 5-Fluorouracil (5-FU), Oxaliplatin, Trifluorothymidine, and Irinotecan. Paclitaxel is the first-line therapy for breast cancer where it binds to microtubules and prevents their disassembly. If microtubules cannot disassemble, they cannot be re-assembling during the process of cell division and cell divisions are inhibited. A low dose of 5-HTR_{2B} antagonist RS-127445 (2 mg/kg/day) was combined with a low dose of paclitaxel (2 mg/kg/alternate days) in the 4T1-luc2 mediated mice breast cancer model. It was found that this combination produces a much better response by reducing tumor growth than any of them alone (FIG. 16A). 5-Fluorouracil is one of the first-line medications for colorectal carcinoma. It is an analog of uracil and it gets incorporated in the place of uracil during mRNA synthesis by transcription and the whole transcription machinery halts. A low dose of 5-HTR_{2B} antagonist RS-127445 (2 mg/kg/day) was combined with a low dose of 5-Fluorouracil (10 mg/kg/alternate days) in the MC38 cell line mediated mice colon cancer orthotropic model. It was found that this combination produces a much better response by reducing tumor growth than any of them alone (FIG. 16B). Oxaliplatin is a non-targeted anti-neoplastic drug that is used alone or in combination with other drugs in colon cancer. Oxaliplatin causes DNA damage to the cell by forming platinum adducts with the DNA and subsequently halting DNA replication, and transcription and causing reduced cell divisions and apoptosis. The combination of low dose Oxaliplatin (4 mg/kg/day for 5 days) with low dose 5-HTR_{2B} antagonist RS-127445 (2 mg/kg/day) in mice subcutaneous colon cancer model significantly enhanced the efficacy of oxaliplatin or RS-127445 alone (FIG. 16C). Irinotecan, an FDA-approved chemotherapeutic agent to be used in colorectal cancer that binds to the topoisomerase I-DNA complex, and this ternary complex stalls the movement of the replication fork and inhibits DNA replication. The combination of low dose Irinotecan (15 mg/kg/day for 5 days) with low dose 5-HTR_{2B} antagonist RS-127445 (2 mg/kg/day) in mice subcutaneous colon cancer model significantly enhanced the efficacy of irinotecan or RS-127445 alone (FIG. 16D). Trifluridine is an approved drug to be used as first-line therapy for CRC. It is a fluorinated pyrimidine nucleoside that inhibits thymidylate synthase irreversibly and also inhibits specific DNA polymerases, necessary for the conversion of dUMP to dTMP in the process of DNA synthesis. We have also combined low dose RS-127445 (2 mg/kg/day) with low dose trifluorothymidine (20 mg/kg/day). We have not found any significant alterations in the efficacy of the trifluorothymidine (FIG. 16E). Altogether, the combination of 5-HTR_{2B} antagonists with different many clinically approved chemotherapeutic agents even at low doses enhanced their efficacy compared to alone. This not only gives a new paradigm to enhance the efficacy of chemotherapeutic agents in treating cancers but also will reduce the chemotherapy-associated toxicities to some extent as they are used in quite low doses. Though there are a few more tests needed to be performed to explore the mechanism of action of 5-HTR_{2B} antagonists in the combination of the chemotherapeutic agents. Our best guess will be as the 5-HTR_{2B} antagonism promotes the antigen-specific CTL response and these chemotherapeutic agents affect the division and proliferation of cancer cells. As this combination engages two arms to prevent tumor growth by suppressing both cancer cell proliferation and antigen-dependent killing of the cancer cells, this combinatorial approach can be very effective to prevent colorectal cancer growth.

1-29. (canceled)

30. An anti-cancer composition comprising:
a. 5-HTR_{2B} antagonistic compound, and
b. an immunomodulatory compound.

31. The anti-cancer composition of claim 30, further including a chemotherapeutic drug.

32. The anti-cancer composition of claim 30, wherein the 5-HTR_{2B} antagonistic compound is selected from the group consisting of Agomelatine, Amisulpride, Aripiprazole, Cariprazine, Clozapine, Cyproheptadine, Sarpogrelate, Lisuride, Tegaserod, RS-127,445, Metadoxine, SDZ SER-082, Promethazine, EGIS-7625, PRX-08066, SB-200,646, SB-204,741, SB-206,553, SB-215,505, SB-228,357, Terguride, LY-266,097, and LY-272,015.

33. The anti-cancer composition of claim 30, wherein the immunomodulatory compound is selected from the group consisting of anti-PD1 mAb, anti-PDL1 mAb, and anti-CTLA4 mAb.

34. The anti-cancer composition of claim 30, wherein the immunomodulatory compound is selected from the group consisting of Pembrolizumab, Nivolumab, Cemiplimab, JTX-4014, Sintilimab, Spartalizumab, Camrelizumab, Tislelizumab, Toripalimab, Dostarlimab, INCMGA00012, AMP-224, AMP-514, CD279, Atezolizumab, Avelumab, Durvalumab, CD274, B7-H1, KN035, cosibelimab, AUNP12, CA-170, BMS-986189, Ipilimumab, Tremelimumab, and CD152.

35. The anti-cancer composition of claim 33, wherein the immunomodulatory compound is selected from the group consisting of Pembrolizumab, Nivolumab, Cemiplimab, JTX-4014, Sintilimab, Spartalizumab, Camrelizumab, Tislelizumab, Toripalimab, Dostarlimab, INCMGA00012, AMP-224, AMP-514, and CD279.

36. The anti-cancer composition of claim 33, wherein the immunomodulatory compound is selected from the group consisting of Atezolizumab, Avelumab, Durvalumab, CD274, B7-H1, KN035, cosibelimab, AUNP12, CA-170, and BMS-986189.

37. The anti-cancer composition of claim 30, wherein the immunomodulatory compound is selected from the group consisting of Ipilimumab, Tremelimumab, and CD152.

38. The anti-cancer composition of claim 31, wherein the chemotherapeutic drug is selected from the group consisting of 5-fluorouracil (5-FU), paclitaxel, oxaliplatin, trifluorothymidine, and irinotecan.

39. The anti-cancer composition of claim 30, wherein the 5-HTR_{2B} antagonistic compound is present in a range of 30% to 70% by weight of the composition.

40. The anti-cancer composition of claim 30, wherein the 5-HTR_{2B} antagonistic compound is present in a range of 10% to 40% by weight of the composition.

41. The anti-cancer composition of claim 30, wherein the 5-HTR_{2B} antagonistic compound is present in a range of 40% to 60% by weight of the composition.

42. The anti-cancer composition of claim 30, wherein the 5-HTR_{2B} antagonistic compound is present in a range of 15% to 50% by weight of the composition.

43. The anti-cancer composition of claim 30, wherein the 5-HTR_{2B} antagonistic compound is present in a range of 5% to 30% by weight of the composition.

44. The anti-cancer composition of claim 30, wherein the 5-HTR_{2B} antagonistic compound is present in a range of 1 to 40 mg/kg of body weight of an animal being treated.

45. The anti-cancer composition of claim 30, wherein the immunomodulatory compound is present in a range of 1 to 20 mg/kg of body weight of an animal being treated.

46. The anti-cancer composition of claim 31, wherein the chemotherapeutic drug, 5-FU is present in a range of 5 to 50 mg/kg of body weight of an animal being treated.

47. The anti-cancer composition of claim 31, wherein the chemotherapeutic drug, Paclitaxel is present in a range of 2 to 10 mg/kg of body weight of an animal being treated.

48. The anti-cancer composition of claim 31, wherein the chemotherapeutic drug is Oxaliplatin and is present in a range of 4 to 10 mg/kg of body weight of an animal being treated.

49. The anti-cancer composition of claim 31, wherein the chemotherapeutic drug is Irinotecan and is in the range of 15 to 50 mg/kg of body weight of an animal being treated.

50. A pharmaceutical composition comprising the composition of claim 30 and a pharmaceutically acceptable excipient.

51. The pharmaceutical composition of claim 50, wherein the composition is selected from the group consisting of oral composition, intradermal composition transdermal composition, parenteral composition, intramuscular composition, intrathecal composition, topical composition, intravaginal composition, intravesical composition, intracisternal composition, and rectal composition.

52. The pharmaceutical composition of claim 50, wherein the composition is a solid, a semi-solid, a lyophilized powder, a liquid dosage form, or an aerosol.

53. The pharmaceutical composition of claim 50, which is configured for administering at a dose range of 1 to 100 mg/kg body weight of a patient.

54. A method of treating a cancer of epithelial origin in a patient comprising administering to the patient an effect amount of a pharmaceutical composition of claim 50.

55. The method of claim 54, wherein the cancer of epithelial origin is selected from the group consisting of melanoma, breast cancer, and colon adenocarcinoma.

56. The method of claim 54, wherein the treatment further includes administration of a chemotherapeutic drug.
